(19) United States
(12) Patent Application Publication
(10) Pub. No.: US 2025/0228907 A1
(43) Pub. Date: Jul. 17, 2025

(54) COMPOSITIONS AND METHODS FOR TREATING METACHROMATIC LEUKODYSTROPHY DISEASE AND RELATED DISORDERS

(71) Applicant: The Children's Hospital of Philadelphia, Philadelphia, PA (US)

(72) Inventors: Stefano Rivella, Philadelphia, PA (US); Lucas Tricoli, Collegeville, PA (US)

(21) Appl. No.: 18/700,122

(22) PCT Filed: Nov. 3, 2022
(86) PCT No.: PCT/US2022/079211

§ 371 Date: Apr. 10, 2024

Related U.S. Application Data
(60) Provisional application No. 63/275,606, filed on Nov. 4, 2021.

(51) Int. Cl. A61K 35/76; A61K 38/19; A61K 38/44; A61K 38/46; A61K 38/47; A61K 48/00; C07K 14/52; C12N 9/02; C12N 9/16; C12N 9/24; C12N 15/86

(52) U.S. Cl. CPC A61K 35/76; A61K 48/005; C07K 14/52; C12N 9/0051; C12N 9/0089; C12N 9/16; C12N 9/2402; C12N 15/86; A61K 38/19; A61K 38/446; A61K 38/465; A61K 38/47; C12N 2740/16043; C12N 2830/40; C12Y 108/99; C12Y 115/01001; C12Y 301/06001; C12Y 302/01046; C12Y 310/01001

(57) ABSTRACT

The invention provides compositions and methods for the treatment of lysosomal storage diseases such as metachromatic leukodystrophy and related disorders. A significant unmet clinical need still exists with the current clinical lentiviral vectors due to the high integration requirement and currently insufficient levels of ARSA expression to correct onset of early symptomatic disease. Methods and compositions for producing a protein such as sulfatase and for treating a disease or disorder such as metachromatic leukodystrophy and related disorders are disclosed.

This application claims priority under 35 U.S.C. § 119 (e) to U.S. Provisional Patent Application No. 63/275,606, filed Nov. 4, 2021. The foregoing application is incorporated by reference herein.

### FIELD OF THE INVENTION

The present invention relates to the field of therapeutics. More specifically, the invention provides compositions and methods for the treatment of lysosomal storage diseases such as metachromatic leukodystrophy and related disorders.

### BACKGROUND OF THE INVENTION

Several publications and patent documents are cited throughout the specification in order to describe the state of the art to which this invention pertains. Each of these citations is incorporated herein by reference as though set forth in full. Metachromatic leukodystrophy (MLD) is an autosomal recessive lysosomal storage disease (LSD) characterized by a mutation of the enzyme arylsulfatase A (ARSA) most often affecting children in the late infantile stage (LI) (less than 30 months of age at onset), which is the most rapidly progressive form of the disease. Utilizing ex vivo hematopoietic stem cell (HSC) gene therapy, a functional copy of the ARSA gene has the potential for restoring proper enzyme activity in MLD and has preliminarily demonstrated successful treatment in clinical trials (Biffi, et al., Science (2013) 341(6148):1233158). Current approaches for MLD rely on the use of adeno-associated virus vectors (AAVs) or lentiviral vectors (LVs), which for AAV may not sustain high level of gene expression in neurological disorders or for LV requires multiple integrations to prevent the development of the disease. Further, AAVs also exhibit significant toxicity, morbidity and mortality. Thus, a significant unmet clinical need still exists with the current clinical LV due to the high integration requirement and currently insufficient levels of ARSA expression to correct onset of early symptomatic disease.

### SUMMARY OF THE INVENTION

In accordance with one aspect of the instant invention, nucleic acid molecules and vectors, particularly viral vectors such as lentiviral vectors, are provided. In a particular embodiment, the nucleic acid or vector comprises a nucleic acid molecule comprising any one or more of: i) a 5′ long terminal repeat (LTR) and a 3′ LTR (e.g., at least one of the LTR may be self-inactivating); ii) at least one polyadenylation signal; iii) a EF1-alpha promoter or CD68 promoter; iv) an insulator (e.g., an ankyrin insulator element (Ank) and/or foamy virus insulator); v) a Woodchuck Post-Regulatory Element (WPRE) (e.g., wherein the WPRE is 3′ of the 3′LTR); and/or vi) a sequence encoding a protein (e.g., a therapeutic protein) such as a sulfatase (e.g., ARSA) and/or FGE. The instant invention also encompasses cells (e.g., hematopoietic stem cells, hematopoietic progenitor cells, bone marrow cells, microglia, macrophage, and/or fibroblasts) and viral particles comprising the nucleic acid or vector (e.g., lentiviral vector) of the instant invention. Compositions comprising the nucleic acid or vector (e.g., lentiviral vector) or viral particles are also encompassed by the instant invention. The compositions may further comprise a pharmaceutically acceptable carrier.

In accordance with another aspect of the instant invention, methods of inhibiting, treating, and/or preventing a disease or disorder in a subject are provided. In certain embodiments, the disease or disorder is multiple sulfatase deficiency (MSD), metachromatic leukodystrophy (MLD) and/or a sulfatase deficiency. In certain embodiments, the disease or disorder is ALD, MPS IIIA, Krabbe, or ALS. In a particular embodiment, the method comprises administering a nucleic acid or viral vector or viral particle of the instant invention to a subject in need thereof. In a particular embodiment, the method comprises an ex vivo therapy (e.g., autologous bone marrow cells, hematopoietic stem cells, microglia, macrophage, or fibroblasts) utilizing a nucleic acid, viral vector, or viral particle of the instant invention. The nucleic acid, viral vector, or viral particle may be in a composition with a pharmaceutically acceptable carrier.

### BRIEF DESCRIPTIONS OF THE DRAWINGS

FIG. 1 provides schematics of certain vectors.

FIG. 2A provides a graph of the mean fluorescence index of HePG2 cells transduced with the indicated vectors. Outlined vectors comprise insulators. FIG. 2B provides a graph of the mean fluorescence index of MEL cells transduced with the indicated vectors. Outlined vectors comprise insulators.

FIG. 3A provides a graph of ARSA activity per vector copy number (VCN) in transduced human MLD fibroblasts for the following vectors: 1: PawMut6; 2: EAAWP-UTR+; 3: EAAWP-UTR−; 4: TCO-EAAWP-UTR+; 5: TCO-EAAWP-UTR−; 6: TCO-EAFWP-UTR−; and 7: TCO-AEAFWP-UTR−. FIG. 3B provides a graph of ARSA activity normalized to VCN in transduced human MLD fibroblasts with the indicated vectors. N=3.

FIG. 4 provides Western blot analyses of ARSA from MLD fibroblasts transduced with PAWMut6, TCO-EAFWP-UTR−, TCO-AEAFWP-UTR−, and TCO-EAAWP-UTR+. Beta-actin is provided as a control.

FIGS. 5A and 5B provide graphs of the activity of ARSA in fibroblasts (FIG. 5A) and microglia (FIG. 5B) transduced with PawMut6 or TCO-EAFWP-UTR-before and after formylglycine-generating enzyme (FGE) transduction.

FIG. 6A shows ARSA activity in mouse hematopoietic stem cells (HSC) transduced with PawMut6 or TCO-AEAFWO-UTR− at 1 and 2 weeks after transduction. FIG. 6B shows ARSA activity per VCN in mouse HSCs transduced with PawMut6, TCO-EAFWP-UTR−, TCO-AEAFWO-UTR− or TCO-EAAWP-UTR+.

FIGS. 7A and 7B show the ARSA activity (FIG. 7A) and ARSB activity (FIG. 7B) in MSD cell lines transduced with the indicated vectors.

FIG. 8 provides a Western blot analysis of FGE vector transduced MSD fibroblasts. 42 kD is uncleaved form and 38 kD is cleaved form.

FIG. 9 provides Western blot analysis of ARSA in fibroblast or microglia media from cells transduced with PawMut6 or TCO-EAFWP-UTR−.

FIG. 10A provides a Western blot analysis of cell lystaes and extracellular vesicles (EV) from cells transduced with PawMut6 or TCO-EAFWP-UTR−. FIG. 10B provides graphs of ARSA activity in cells and EVs from cells transduced with PawMut6 or TCO-EAFWP-UTR−.

FIGS. 11A-11C provide nucleic acid sequences of certain vector components and elements.

FIGS. 12A-12J provide a nucleic acid sequence of AEAFWP (SEQ ID NO: 17).

### DETAILED DESCRIPTION OF THE INVENTION

Herein, vectors expressing higher levels of ARSA at a single integration are provided which will reduce the chances of genotoxicity, be curative in symptomatic MLD patients, and reduce the requirement for full myeloablation. The only clinical vector (CV) that has been utilized to treat MLD patients, PawMut6, includes the human ARSA gene under control of the human PGK promoter and includes, in the integrating transcriptional unit, the viral sequence WPRE to increase titer and mRNA translation (Biffi, et al., Science (2013) 341(6148):1233158). To increase expression of ARSA at single integration level, several lentiviral vectors were generated that include the ARSA gene sequences and viral insulators to optimize ARSA expression and enhance safety in virally transduced cell lines. The human Elongation Factor 1 alpha (EF1-alpha) promoter is a much stronger promoter across a range of different cell lines compared to the human Phosphoglycerate Kinase (PGK) promoter. The different variations of the ARSA gene are with and without the 5′ and 3′ untranslated regions (UTR+ or UTR−) and an additional codon modified version to distinguish from the endogenous ARSA (TCO). An ankyrin and/or foamy insulator has been incorporated to explore further reduction in the possibility of any genotoxicity caused by the vectors. The Woodchuck Hepatitis Virus Posttranscriptional Regulatory Element (WPRE) has been proven to enhance the performance of viral vectors. However, to remove the WPRE from the vector integrating sequence, it was placed directly after the 3′-self inactivating LTR (SIN-LTR) together with a strong polyA signal (pA) (Breda, et al., Mol. Ther. (2021) 29(4):1625-1638).

Using MLD primary patient fibroblast cells, ARSA activity, normalized to vector copy number (VCN), demonstrated that TCO-EAAWP-UTR+, TCO-EAFWP-UTR− and TCO-AEAFWP-UTR− showed more ARSA activity (respectively 2×, 10× and 4×) compared to the currently existing clinical vector PawMut6, with protein analysis by western blot showing a similar trend. The ability of the vectors to secrete more functional ARSA enzyme into the surrounding media in culture of transduced primary MLD patient fibroblast cells was also demonstrated, which is a critical modality for transfer of functional ARSA from microglia to oligodendrocytes. Transgene ARSA secretion has been demonstrated to primarily occur through extracellular vesical secretion, having significant implication for how to approach treatment of MLD. Experiments with the vectors in transduced mouse HSC have demonstrated high levels of transduction with superior levels of ARSA activity. To test for potential toxicity, bone marrow transplants were performed on WT animals with HSCs transduced at up to 13 copies per genome. For the vectors with high vector copy number, tolerability of the vectors has been demonstrated. As such, it is clear that the present invention has provided unexpectedly superior vectors for the expression of ARSA and treating MLD and related disorders.

The data provided herein shows superior gene/protein expression with the vectors of the instant invention compared to PawMut6. Further, the data shows that cells transduced with vectors of the instant invention secrete much more therapeutic protein (ARSA) than cells infected with PawMut6. This is extremely relevant as cells that contain the vector (e.g., transduced bone marrow cells after transplant such as microglia) can correct the bone marrow acting as “drug factories” for the rest of the body including the brain. Herein, it is also demonstrated that ARSA activity (a sulfatase protein) can be further increased by combining the protein formylglycine-generating enzyme (FGE) with ARSA. In addition, FGE/SUMF1 is mutated in MSD (Multiple Sulfatase Deficiency). Therefore, the vector expressing only FGE or a vector expressing FGE and ARSA can be curative in MSD. FGE activates not only ARSA, but several additional sulfatases. Therefore, combination of FGE with other sulfatases can increase the curative potential for many forms of sulfatase deficiency. This also means more sulfatase produced by the curative cells. Microglia in the brain derive from bone marrow cells. Therefore, the vectors of the instant invention can increase the production of curative sulfatase in microglia with less vector and less potential side effects.

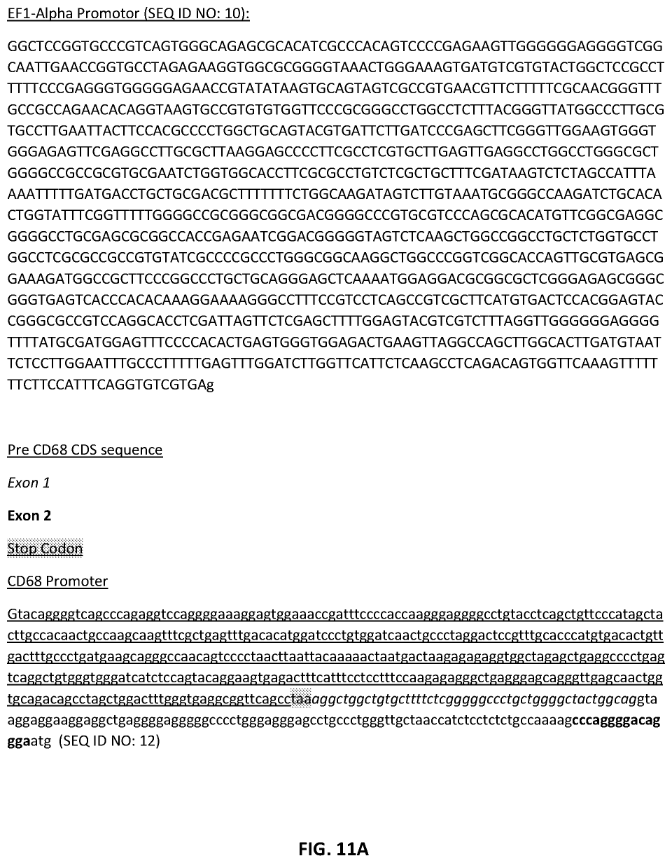

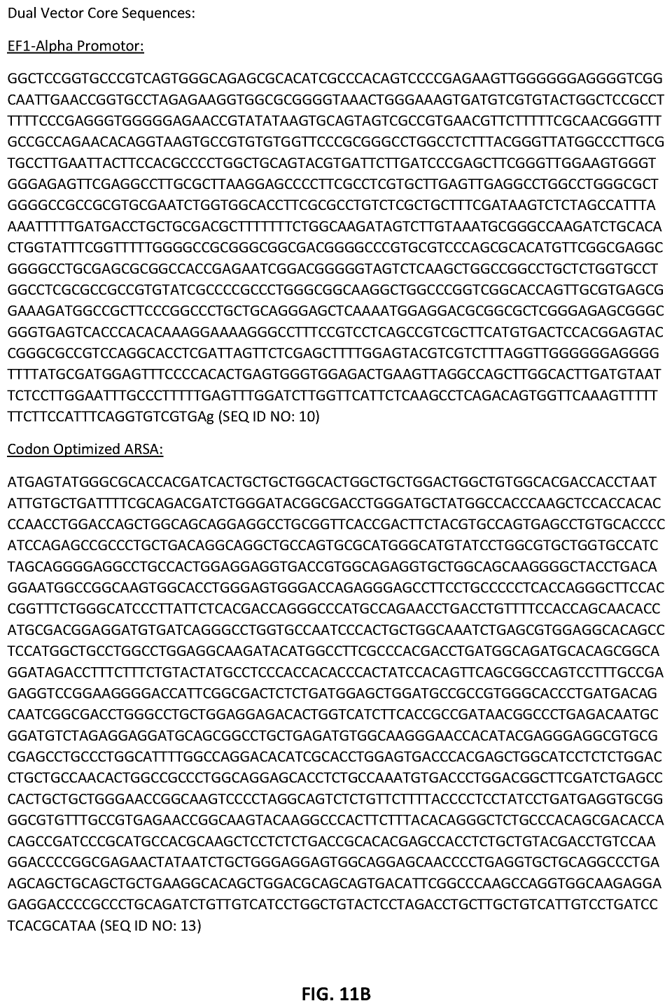

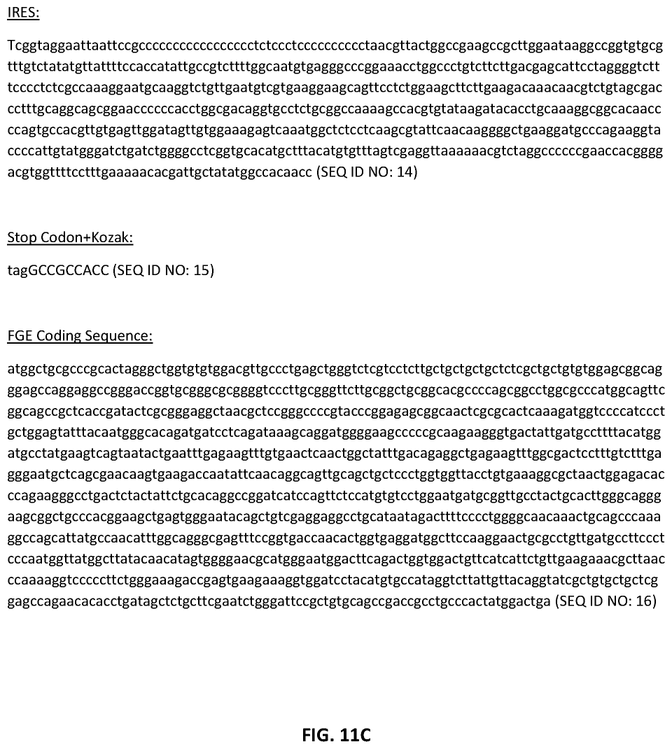

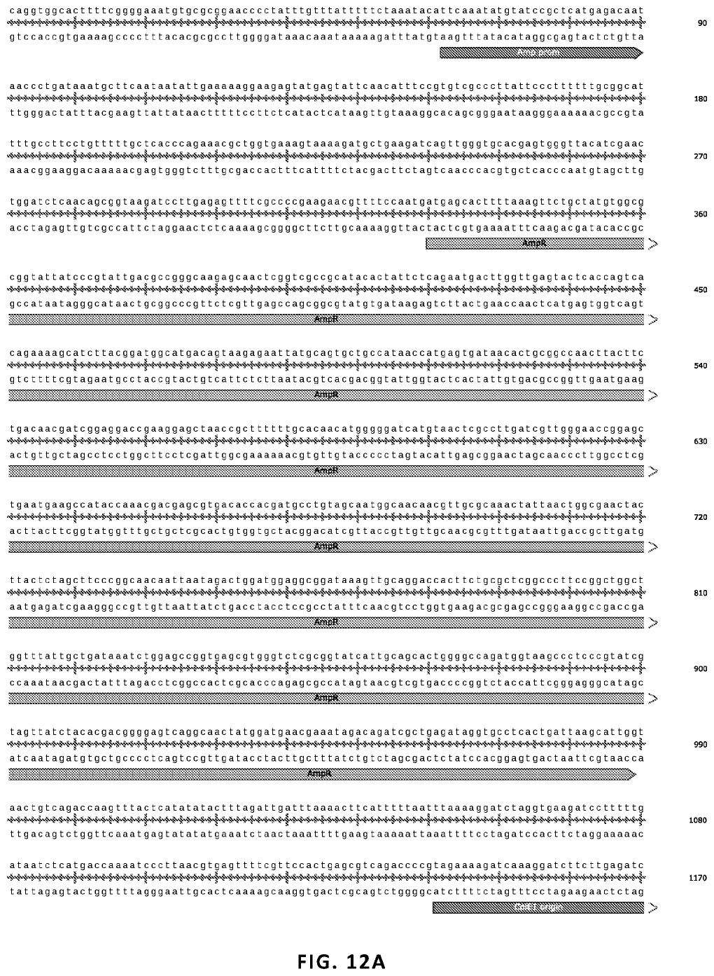

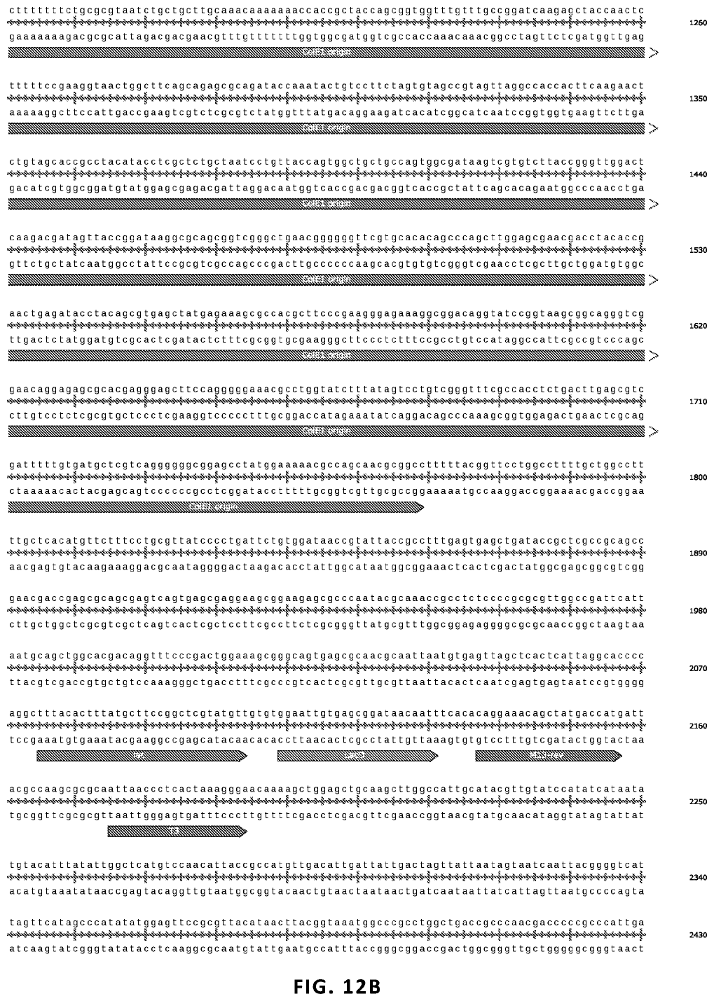

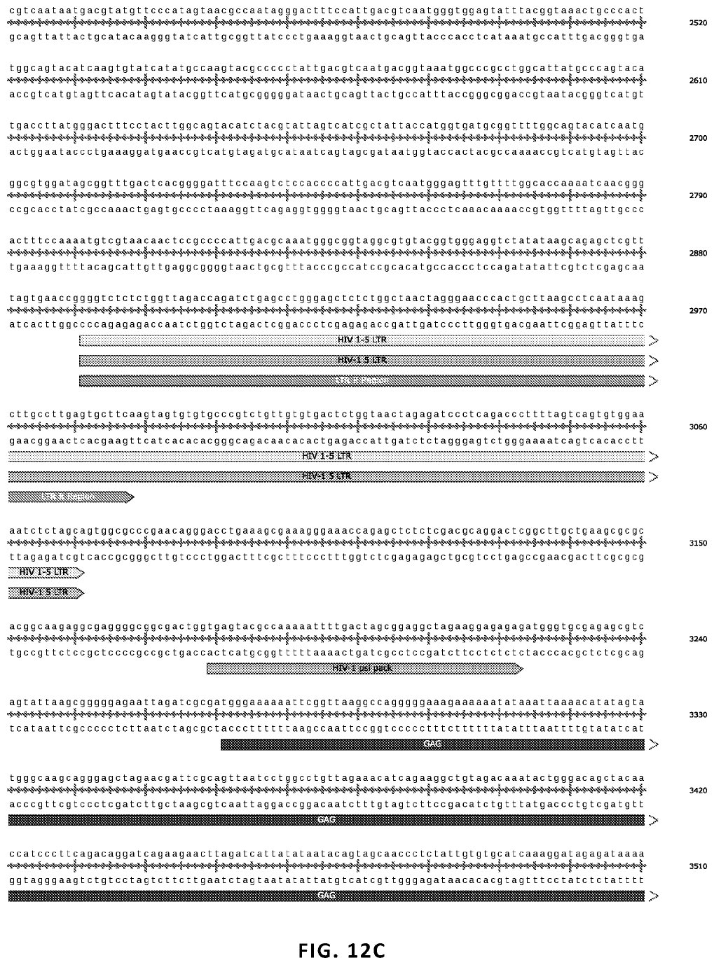

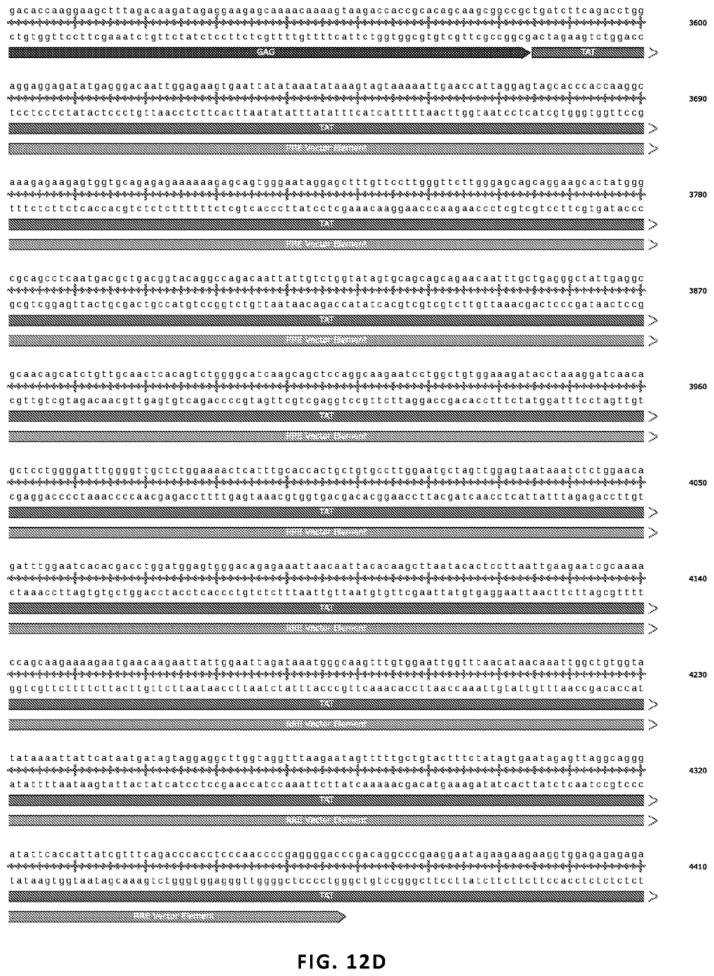

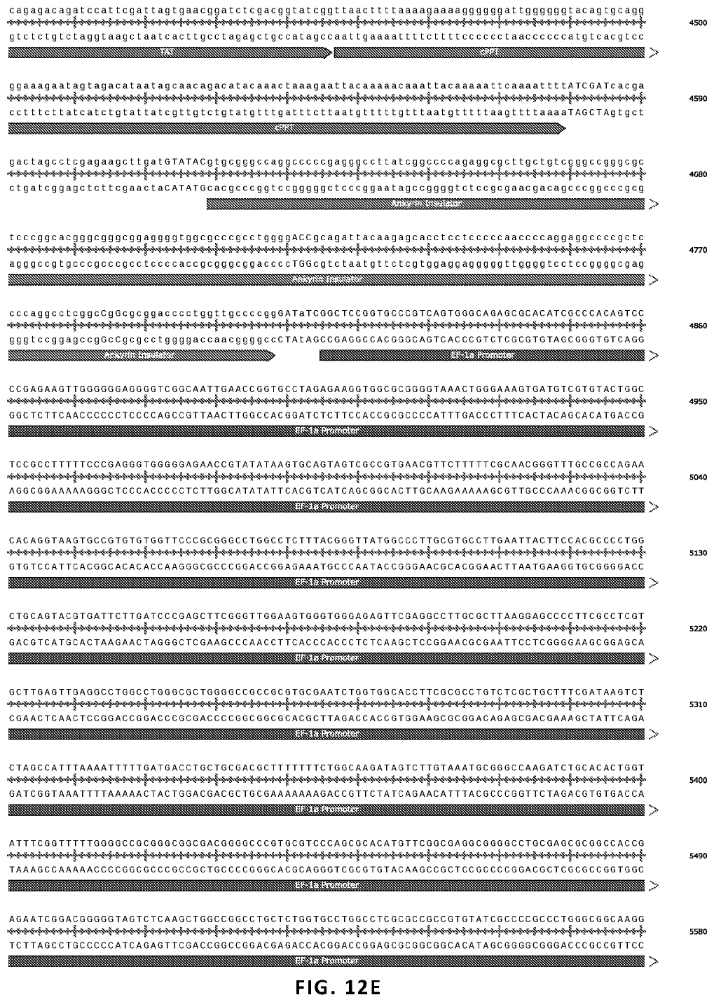

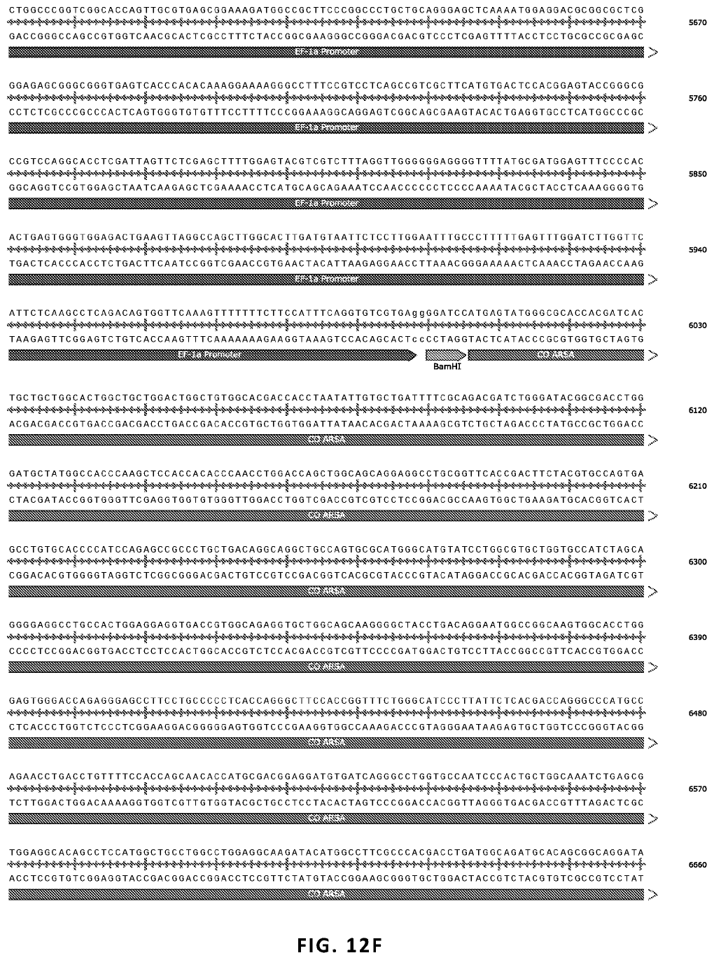

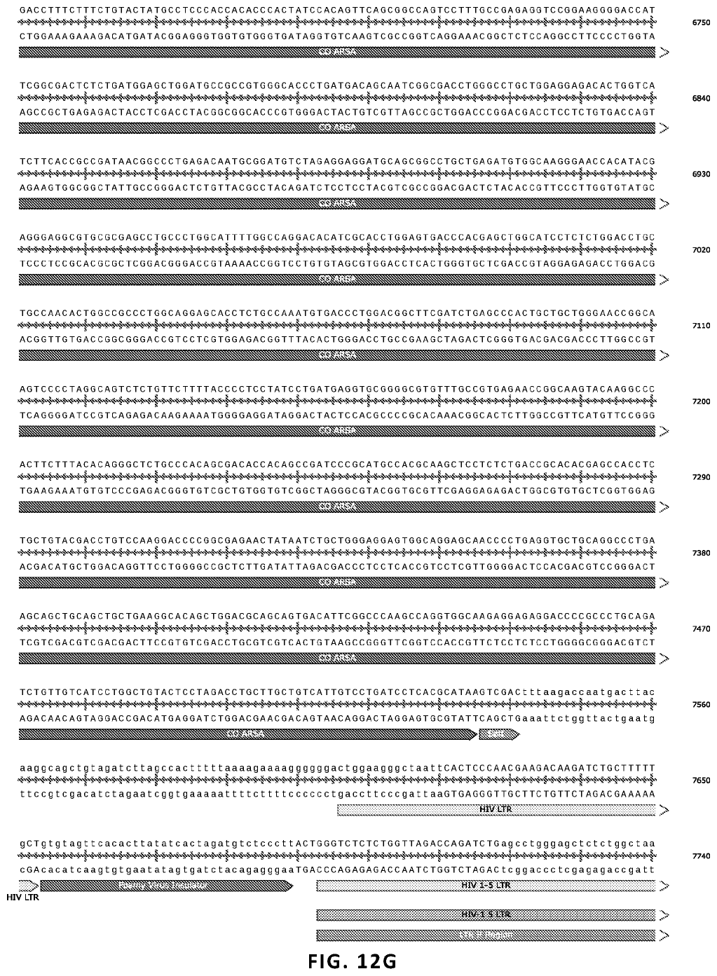

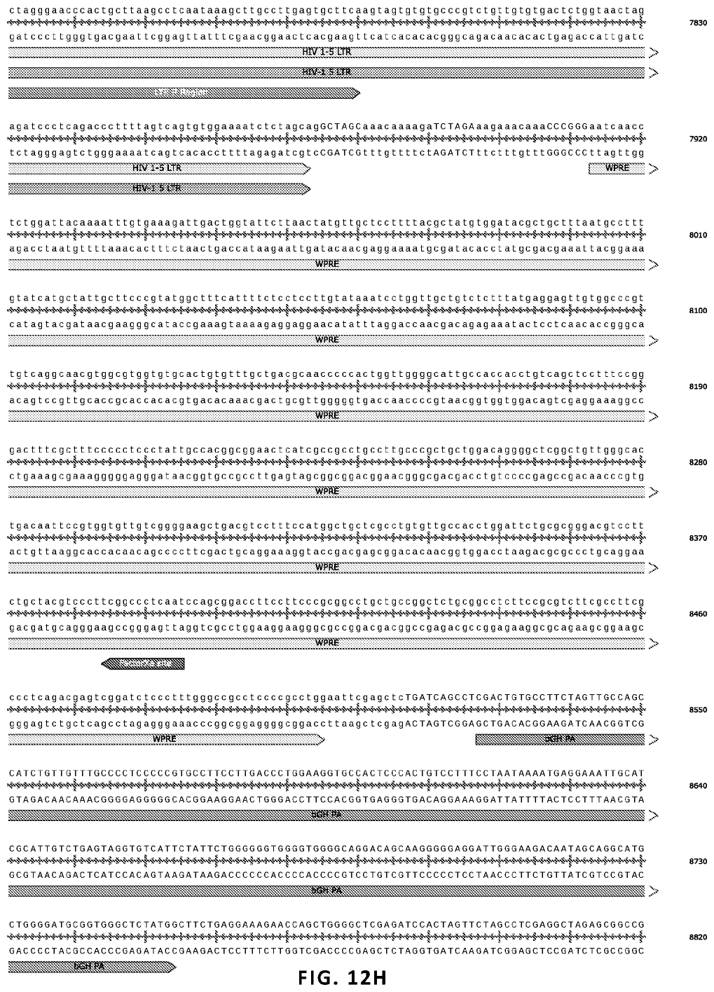

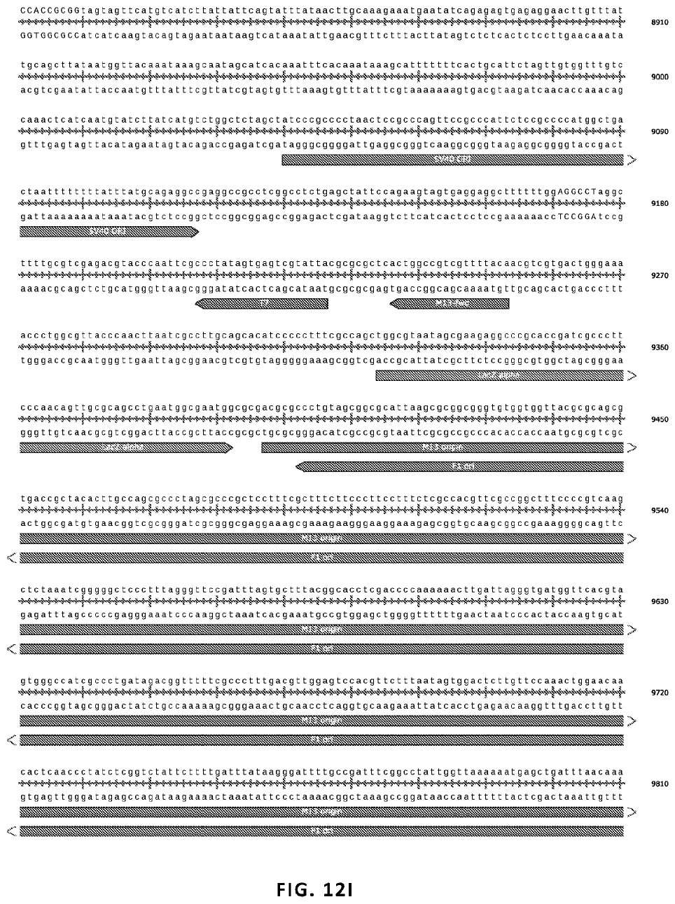

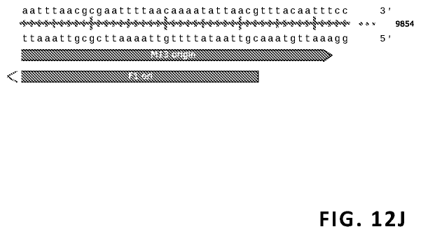

Herein, nucleic acids and viral vectors for the inhibition, prevention, and/or treatment of a disease or disorder such as multiple sulfatase deficiency (MSD), metachromatic leukodystrophy (MLD) and/or a sulfatase deficiency are provided. Generally, the nucleic acids of the instant invention will be a vector, particularly a viral vector. In certain embodiments, the viral vector comprises: i) a 5′ long terminal repeat (LTR) and a 3′ LTR (particularly, at least one of the LTR (at least the 3′LTR) is self-inactivating; a self-inactivating LTR comprises a deletion or mutation relative to its native sequence that results in it being replication incompetent); ii) at least one promoter (e.g., the elongation factor 1 alpha (EF1-alpha) promoter (e.g., GenBank: J04617.1), CD68 promoter (Gene ID: 968))); and iii) a sequence encoding a protein (e.g., a therapeutic protein) such as a sulfatase (e.g., arylsulfatase A (ARSA)). In certain embodiments, the vector further comprises one or more of: at least one polyadenylation signal (e.g., a strong bovine growth hormone poly A tail (e.g., inserted after the WPRE region, if present) increases lentiviral titers (Zaiss, et al. (2002) J. Virol., 76 (14): 7209-19)); an enhancer; at least one insulator element (e.g., an ankyrin insulator element (Ank) and/or foamy virus insulator; particularly, the insulator is within the 3′ LTR); a Woodchuck Post-Regulatory Element (WPRE) (e.g., configured such that the WPRE does not integrate into a target genome); and/or a nucleic acid encoding formylglycine-generating enzyme (FGE). In certain embodiments, the viral vector comprises: i) a 5′ long terminal repeat (LTR) and a self-inactivating 3′ LTR; ii) an EF1-alpha promoter (e.g., operably linked or controlling expression of a protein (e.g., a therapeutic protein) such as the sulfatase (e.g., arylsulfatase A); iii) a sequence encoding a protein (e.g., a therapeutic protein) such as a sulfatase (e.g., arylsulfatase A) and/or a sequence encoding formylglycine-generating enzyme (FGE); v) a Woodchuck Post-Regulatory Element (WPRE); and vi) at least one polyadenylation signal. When the vector comprises ARSA and FGE, the encoding nucleotide sequences may be separated by an IRES (e.g., the IRES in FIG. 11). FIGS. 11 and 12 as well as U.S. Patent Application Publication 2018/0008725; WO 2019/213011; and WO 2020/264488 (these applications are incorporated by reference herein in their entirety), provide viral vectors as well as sequences for certain of the above elements.

In certain embodiments, the viral vector comprises (particularly from 5′ to 3′): i) a 5′ long terminal repeat (LTR); ii) the EF-1 alpha promoter; iii) a sequence encoding a protein (e.g., a therapeutic protein) such as a sulfatase (e.g., arylsulfatase A) and/or a sequence encoding formylglycine-generating enzyme (FGE) (optionally including at least part of or all of the 5′UTR and 3′UTR of the gene); iv) a self-inactivating 3′ LTR comprising an insulator element (e.g., ankyrin insulator element and/or a foamy virus insulator); v) a WPRE; and vi) a polyadenylation signal (e.g., a strong bovine growth hormone poly A tail).

In certain embodiments, the viral vector comprises (particularly from 5′ to 3′): i) a 5′ long terminal repeat (LTR); ii) an insulator element (e.g., an ankyrin insulator element (Ank) and/or foamy virus insulator; iii) the EF-1 alpha promoter; iv) a sequence encoding a protein (e.g., a therapeutic protein) such as a sulfatase (e.g., arylsulfatase A) and/or a sequence encoding formylglycine-generating enzyme (FGE) (optionally including at least part of or all of the 5′UTR and 3′UTR of the gene); v) a self-inactivating 3′ LTR comprising an insulator element (e.g., ankyrin insulator element and/or a foamy virus insulator); vi) a WPRE; and vii) a polyadenylation signal (e.g., a strong bovine growth hormone poly A tail).

While the nucleic acid molecules and viral vectors of the instant invention are generally exemplified herein for the expression of ARSA, the nucleic acid molecules and viral vectors of the instant invention can contain any nucleic acid sequence to be expressed (e.g., encode a therapeutic protein to treat a particular disease or disorder (e.g., a protein that is defective, mutant, or missing as a causative agent of the disease or disorder)). In certain embodiments, the viral vector comprises a sequence encoding ATP binding cassette subfamily D member 1 (ABCD1; GenBank Gene ID: 215; e.g., GenBank Accession Nos. NM_000033.4 and NP_000024.2) (e.g., in place of the sequence encoding the sulfatase). Such nucleic acids and viral vectors can be used for the inhibition, prevention, and/or treatment of adrenoleukodystrophy (ALD).

In certain embodiments, the nucleic acid molecule or viral vector comprises a sequence encoding N-sulfoglucosamine sulfohydrolase (SGSH; GenBank Gene ID: 6448; e.g., GenBank Accession Nos. NM_000199.5 and NP_000190.1) (e.g., in place of the sequence encoding the sulfatase). Such nucleic acids and viral vectors can be used for the inhibition, prevention, and/or treatment of mucopolysaccharidosis type IIIA (MPS IIIA; also known as Sanfilippo syndrome A).

In certain embodiments, the nucleic acid molecule or viral vector comprises a sequence encoding galactocerebrosidase (GALC; GenBank Gene ID: 2581; e.g., GenBank Accession Nos. NM_000153.4 and NP_000144.2) (e.g., in place of the sequence encoding the sulfatase). The viral vector may further comprise a sequence encoding a sulfatase such as ARSA. Such nucleic acids and viral vectors can be used for the inhibition, prevention, and/or treatment of Krabbe disease.

In certain embodiments, the nucleic acid molecule or viral vector comprises a sequence encoding superoxide dismutase 1 gene (SOD1; GenBank Gene ID: 6647; e.g., GenBank Accession Nos. NM_000454.5 and NP_000445.1) (e.g., in place of the sequence encoding the sulfatase). Such nucleic acids and viral vectors can be used for the inhibition, prevention, and/or treatment of amyotrophic lateral sclerosis (ALS).

Viral vectors include, for example, retroviruses and lentiviruses. In a particular embodiment, the viral vector is a lentiviral vector. The viral vector may comprise one or more (or all) of the modifications listed below. In a particular embodiment, the vector is TCO-EAAWP-UTR+, TCO-EAFWP-UTR−, or TCO-AEAFWP-UTR− or a modified version comprising one or more (or all) of the modifications listed below. The nucleic acid molecule, vector, or viral vector of the instant invention may comprise one or more (or all) of the modifications listed below.

First, in certain embodiments of the instant invention, the sulfatase is arylsulfatase A (ARSA). In certain embodiments, the sulfatase is human. Examples of amino acid and nucleotide sequences of ARSA are provided in GenBank Gene ID: 410 and GenBank Accession Nos. NM_000487.6, NP_000478.3, NM_001085425.3, NP_001078894.2, NM_001085426.3, NP_001078895.2, NM_001085427.3, NP_001078896.2, NM_001085428.3, NP_001078897.1, NM_001362782.2, and NP_001349711.1. In certain embodiments, the ARSA is isoform a. An example of an amino acid sequence of human ARSA is (SEQ ID NO: 1):

| | |
|---|---|
| 1 | MSMGAPRSLL LALAAGLAVA RPPNIVLIFA DDLGYGDLGC YGHPSSTTPN LDQLAAGGLR |
| 61 | FTDFYVPVSL CTPSRAALLT GRLPVRMGMY PGVLVPSSRG GLPLEEVTVA EVLAARGYLT |
| 121 | GMAGKWHLGV GPEGAFLPPH QGFHRFLGIP YSHDQGPCQN LTCFPPATPC DGGCDQGLVP |
| 181 | IPLLANLSVE AQPPWLPGLE ARYMAFAHDL MADAQRQDRP FFLYYASHHT HYPQFSGQSF |
| 241 | AERSGRGPFG DSLMELDAAV GTLMTAIGDL GLLEETLVIF TADNGPETMR MSRGGCSGLL |
| 301 | RCGKGTTYEG GVREPALAFW PGHIAPGVTH ELASSLDLLP TLAALAGAPL PNVTLDGFDL |
| 361 | SPLLLGTGKS PRQSLFFYPS YPDEVRGVFA VRTGKYKAHF FTQGSAHSDT TADPACHASS |
| 421 | SLTAHEPPLL YDLSKDPGEN YNLLGGVAGA TPEVLQALKQ LQLLKAQLDA AVTFGPSQVA |
| 481 | RGEDPALQIC CHPGCTPRPA CCHCPDPHA |

Amino acids 1-20 are a signal peptide and amino acids 21-509 is the mature protein. The ARSA of the instant invention may contain the signal peptide or it may be absent. An example of a nucleotide sequence encoding human ARSA is (SEQ ID NO: 2):

| | |
|---|---|
| 1 | gaggcggaag cgcccgcagc ccggtaccgg ctcctcctgg gctccctcta gcgccttccc |
| 61 | cccggcccga ctccgctggt cagcgccaag tgacttacgc ccccgaccct gagcccggac |
| 121 | cgctaggcga ggaggatcag atctccgctc gagaatctga aggtgccctg gtcctggagg |
| 181 | agttccgtcc cagcccgcgg tctcccggta ctgtcgggcc ccggccctct ggagcttcag |
| 241 | gaggcggccg tcagggtcgg ggagtatttg ggtccggggt ctcagggaag ggcggcgcct |
| 301 | gggtctgcgg tatcggaaag agcctgctgg agccaagtag ccctccctct cttgggacag |
| 361 | acccctcggt cccatgtcca tgggggcacc gcggtccctc ctcctggccc tggctgctgg |
| 421 | cctggccgtt gcccgtccgc ccaacatcgt gctgatcttt gccgacgacc tcggctatgg |
| 481 | ggacctgggc tgctatgggc accccagctc taccactccc aacctggacc agctggcggc |
| 541 | gggagggctg cggttcacag acttctacgt gcctgtgtct ctgtgcacac cctctagggc |
| 601 | cgccctcctg accggccggc tcccggttcg gatgggcatg taccctggcg tcctggtgcc |
| 661 | cagctcccgg gggggcctgc ccctggagga ggtgaccgtg gccgaagtcc tggctgcccg |
| 721 | aggctacctc acaggaatgg ccggcaagtg gcaccttggg gtggggcctg agggggcctt |
| 781 | cctgcccccc catcagggct tccatcgatt tctaggcatc ccgtactccc acgaccaggg |
| 841 | cccctgccag aacctgacct gcttcccgcc ggccactcct tgcgacggtg gctgtgacca |
| 901 | gggcctggtc cccatcccac tgttggccaa cctgtccgtg gaggcgcagc ccccctggct |
| 961 | gcccggacta gaggcccgct acatggcttt cgcccatgac ctcatggccg acgcccagcg |
| 1021 | ccaggatcgc cccttcttcc tgtactatgc ctctcaccac acccactacc ctcagttcag |
| 1081 | tgggcagagc tttgcagagc gttcaggccg cgggccattt ggggactccc tgatggagct |
| 1141 | ggatgcagct gtggggaccc tgatgacagc cataggggac ctggggctgc ttgaagagac |
| 1201 | gctggtcatc ttcactgcag acaatggacc tgagaccatg cgtatgtccc gaggcggctg |
| 1261 | ctccggtctc ttgcggtgtg gaaagggaac gacctacgag ggcggtgtcc gagagcctgc |
| 1321 | cttggccttc tggccaggtc atatcgctcc cggcgtgacc cacgagctgg ccagctccct |
| 1381 | ggacctgctg cctaccctgg cagccctggc tggggcccca ctgcccaatg tcaccttgga |
| 1441 | tggctttgac ctcagccccc tgctgctggg cacaggcaag agccctcggc agtctctctt |
| 1501 | cttctacccg tcctacccag acgaggtccg tggggttttt gctgtgcgga ctggaaagta |
| 1561 | caaggctcac ttcttcaccc agggctctgc ccacagtgat accactgcag accctgcctg |
| 1621 | ccacgcctcc agctctctga ctgctcatga gcccccgctg ctctatgacc tgtccaagga |
| 1681 | ccctggtgag aactacaacc tgctgggggg tgtggccggg gccaccccag aggtgctgca |
| 1741 | agccctgaaa cagcttcagc tgctcaaggc ccagttagac gcagctgtga ccttcggccc |
| 1801 | cagccaggtg gcccggggcg aggaccccgc cctgcagatc tgctgtcatc ctggctgcac |
| 1861 | cccccgccca gcttgctgcc attgcccaga tccccatgcc tgagggcccc tcggctggcc |
| 1921 | tgggcatgtg atggctcctc actgggagcc tgtgggggag gctcaggtgt ctggaggggg |
| 1981 | tttgtgcctg ataacgtaat aacaccagtg gagacttgca gatgtgacaa ttcgtccaat |
| 2041 | cctggggtaa tgctgtgtgc tggtgccggt cccctgtggt acgaatgagg aaactgaggt |
| 2101 | gcagagaggt tcaggacttg tacaagatca cccagccaga aagaggttgg gctgggattt |
| 2161 | gaaccctggt gtcgtggctc tggaagctgc cctggcgcct tggtgatctg cgtgggtcag |
| 2221 | tgcacacagg cacacgtcag cctcaaggac atgggcacat ctgttcacag gagcagcgcc |
| 2281 | acgtgccttt gagtgccagg aacggggtgg gagggtggga gggtgtgagg gccagaagac |
| 2341 | tcagaagatg caaagtgcct gagagagacg ggatattccc ccagaagaag cattcttaga |
| 2401 | gacacaggca ctggacctcc ttggttctta taagaaacct gtctgaagct gggtgatgag |
| 2461 | ttgcacactc caggtggggc taaggggcct ggagcccctg ctggctccta ggaaggcaca |
| 2521 | gcagcaggcc ctgagacggc tcctctgggg cccctccacc ctcccaggcc tctgcatttc |
| 2581 | acctgtgccc acacttctgt ctcctgcctt caccttttga cccactacta acgattctcc |
| 2641 | acccagcaga caaagtgatc tcttaaaaat atctgttggc tgggcacggt ggctcacgcc |
| 2701 | tgtaatccca gcactttagg aagccgaggc gggtggatca cctgaggtcg ggagttcgag |
| 2761 | accagcctga ccaacatgga gaaaccccat ctctactaaa aatacaaaat tagccaggtg |
| 2821 | tagtggtgca tccctgtaat cccagctact tgggagtctg aggctggaga atcacttgaa |
| 2881 | cctgggaggc ggtggttgca gtgagccgag atcgcaccat tgcactccag cctgggcaac |
| 2941 | aagagaaaaa ctctgtctca aaaaacaaaa aatctgttag gctgcacacg gcgattcact |
| 3001 | cctgtattcc cagtgctttg ggaggctgag gtgagaggat gcctgaggcc aggaattcag |
| 3061 | accagcctgg gcaacatagt gagaccccag ctctaaagat ttgtttttgt tttttttttt |
| 3121 | tttttttttt tttttttttt tttttttttg agacggagtc tcgctctgtc gcccaggcta |
| 3181 | gagtgcagtg gtaccatctc cgctcactgc aacctccgcc tcccgggttc cagggattct |
| 3241 | cctgcctcag cctccctagt agctggaact acaggtgtgt gctgccatgc ccagctaatt |
| 3301 | tttttttatt taatagagac aagatttcac catgttggcc aggctggtct caaactcctg |
| 3361 | acctcaggtg atccacccgc ctcagcctcc caaagtgctg ggattacagg tgtgaaccac |
| 3421 | cacacctggc caacaatatt tgttttaatt agccaggcgt ggtagcattt gtcctagcaa |
| 3481 | tttgggaggt tgaggtggga gaatcacttc agcccactag gtcgaggctg tagtgagcta |
| 3541 | taattgtacc actgcactcc agcctcgggg acagagtgag accctgtctg caaataaaca |
| 3601 | aataaaacat caggctgggc ttgagcatct attcctgctc aaaatttcgc aggcttctca |
| 3661 | gaagaaaatc caaacccctt acagtgaccc agtttgccct tgaggcctcc acccacaccc |
| 3721 | ccttcccccc agtcttaggg ggtggcctgg ctgttccctt caacggcaac gctctgcctc |
| 3781 | cattgttggc ctcctctgca gggagggact gtctgagcac ctgcccgtgt ctgtgcagca |
| 3841 | tggcacactg acgtcaggcc cacgtgcatg cccaggtggc cagtcacacg ccaggtgctc |
| 3901 | cctcagtgtt ggccaagtga gaggagcaca ccttccgggc gttcagacac ctccccgtgg |
| 3961 | cagacaccgt tcgttgctac caaacagcca cctccttcct aatgggctcc catttttcag |
| 4021 | tgctgggcaa aggtcccttg atcttggagt tgcagcctct ttctctccaa ggagggcggt |
| 4081 | gaccagcctg agccagtcaa tccagtgatt ggttcaggag tagcctgtga ccaggagtcc |
| 4141 | tggtagtgaa cgactggggc agccctgggg gtgaggacct tgcgcagccg tcacaggccc |
| 4201 | tgattggaca ctgggcagct gctaacccag tgtctccagc tgcctacctg gagagctcca |
| 4261 | agcgtaagaa aataaaccct gcctgttgaa gcca |

Nucleotides 374-1903 translate into the above amino acid sequence and also encodes for ASRA. In certain embodiments, the nucleotide sequence encoding human ASRA comprises nucleotides 374-1903. In certain embodiments, the nucleotide sequence encoding human ASRA comprises the nucleotide sequence for ARSA provided in FIG. 11 or 12.

In certain embodiments, the nucleic acid molecule encoding the sulfatase (e.g., arylsulfatase A (ARSA)) is codon optimized and/or codon modified from the wild-type nucleotide sequence (e.g., encode the same amino acid sequence but with different codon usage) (see, e.g., FIG. 11B). In certain embodiment, the amino acid or nucleotide sequence of ARSA has at least 80, 81, 82, 83, 84, 85, 86, 87, 88, 89, 90, 91, 92, 93, 94, 95, 96, 97, 98, or 99% identity with any of the above sequences.

Second, in certain embodiments, the nucleic acid molecule or vector comprises a sequence encoding ABCD1. An example of a nucleotide sequence encoding human ABCD1 is (SEQ ID NO: 3):

| | |
|---|---|
| 1 | ATGCCGGTGCTCTCCAGGCCCCGGCCCTGGCGGGGGAACACGCTGAAGCG |
| 51 | CACGGCCGTGCTCCTGGCCCTCGCGGCCTATGGAGCCCACAAAGTCTACC |
| 101 | CCTTGGTGCGCCAGTGCCTGGCCCCGGCCAGGGGTCTTCAGGCGCCCGCC |
| 151 | GGGGAGCCCACGCAGGAGGCCTCCGGGGTCGCGGCGGCCAAAGCTGGCAT |
| 201 | GAACCGGGTATTCCTGCAGCGGCTCCTGTGGCTCCTGCGGCTGCTGTTCC |
| 251 | CCCGGGTCCTGTGCCGGGAGACGGGGCTGCTGGCCCTGCACTCGGCCGCC |
| 301 | TTGGTGAGCCGCACCTTCCTGTCGGTGTATGTGGCCCGCCTGGACGGAAG |
| 351 | GCTGGCCCGCTGCATCGTCCGCAAGGACCCGCGGGCTTTTGGCTGGCAGC |
| 401 | TGCTGCAGTGGCTCCTCATCGCCCTCCCTGCTACCTTCGTCAACAGTGCC |
| 451 | ATCCGTTACCTGGAGGGCCAACTGGCCCTGTCGTTCCGCAGCCGTCTGGT |
| 501 | GGCCCACGCCTACCGCCTCTACTTCTCCCAGCAGACCTACTACCGGGTCA |
| 551 | GCAACATGGACGGGCGGCTTCGCAACCCTGACCAGTCTCTGACGGAGGAC |
| 601 | GTGGTGGCCTTTGCGGCCTCTGTGGCCCACCTCTACTCCAACCTGACCAA |
| 651 | GCCACTCCTGGACGTGGCTGTGACTTCCTACACCCTGCTTCGGGCGGCCC |
| 701 | GCTCCCGTGGAGCCGGCACAGCCTGGCCCTCGGCCATCGCCGGCCTCGTG |
| 751 | GTGTTCCTCACGGCCAACGTGCTGCGGGCCTTCTCGCCCAAGTTCGGGGA |
| 801 | GCTGGTGGCAGAGGAGGCGCGGCGGAAGGGGGAGCTGCGCTACATGCACT |
| 851 | CGCGTGTGGTGGCCAACTCGGAGGAGATCGCCTTCTATGGGGGCCATGAG |
| 901 | GTGGAGCTGGCCCTGCTACAGCGCTCCTACCAGGACCTGGCCTCGCAGAT |
| 951 | CAACCTCATCCTTCTGGAACGCCTGTGGTATGTTATGCTGGAGCAGTTCC |
| 1001 | TCATGAAGTATGTGTGGAGCGCCTCGGGCCTGCTCATGGTGGCTGTCCCC |
| 1051 | ATCATCACTGCCACTGGCTACTCAGAGTCAGATGCAGAGGCCGTGAAGAA |
| 1101 | GGCAGCCTTGGAAAAGAAGGAGGAGGAGCTGGTGAGCGAGCGCACAGAAG |
| 1151 | CCTTCACTATTGCCCGCAACCTCCTGACAGCGGCTGCAGATGCCATTGAG |
| 1201 | CGGATCATGTCGTCGTACAAGGAGGTGACGGAGCTGGCTGGCTACACAGC |
| 1251 | CCGGGTGCACGAGATGTTCCAGGTATTTGAAGATGTTCAGCGCTGTCACT |
| 1301 | TCAAGAGGCCCAGGGAGCTAGAGGACGCTCAGGCGGGGTCTGGGACCATA |
| 1351 | GGCCGGTCTGGTGTCCGTGTGGAGGGCCCCCTGAAGATCCGAGGCCAGGT |
| 1401 | GGTGGATGTGGAACAGGGGATCATCTGCGAGAACATCCCCATCGTCACGC |
| 1451 | CCTCAGGAGAGGTGGTGGTGGCCAGCCTCAACATCAGGGTGGAGGAAGGC |
| 1501 | ATGCATCTGCTCATCACAGGCCCCAATGGCTGCGGCAAGAGCTCCCTGTT |
| 1551 | CCGGATCCTGGGTGGGCTCTGGCCCACGTACGGTGGTGTGCTCTACAAGC |
| 1601 | CCCCACCCCAGCGCATGTTCTACATCCCGCAGAGGCCCTACATGTCTGTG |
| 1651 | GGCTCCCTGCGTGACCAGGTGATCTACCCGGACTCAGTGGAGGACATGCA |
| 1701 | AAGGAAGGGCTACTCGGAGCAGGACCTGGAAGCCATCCTGGACGTCGTGC |
| 1751 | ACCTGCACCACATCCTGCAGCGGGAGGGAGGTTGGGAGGCTATGTGTGAC |
| 1801 | TGGAAGGACGTCCTGTCGGGTGGCGAGAAGCAGAGAATCGGCATGGCCCG |
| 1851 | CATGTTCTACCACAGGCCCAAGTACGCCCTCCTGGATGAATGCACCAGCG |
| 1901 | CCGTGAGCATCGACGTGGAAGGCAAGATCTTCCAGGCGGCCAAGGACGCG |
| 1951 | GGCATTGCCCTGCTCTCCATCACCCACCGGCCCTCCCTGTGGAAATACCA |
| 2001 | CACACACTTGCTACAGTTCGATGGGGAGGGCGGCTGGAAGTTCGAGAAGC |
| 2051 | TGGACTCAGCTGCCCGCCTGAGCCTGACGGAGGAGAAGCAGCGGCTGGAG |
| 2101 | CAGCAGCTGGCGGGCATTCCCAAGATGCAGCGGCGCCTCCAGGAGCTCTG |
| 2151 | CCAGATCCTGGGCGAGGCCGTGGCCCCAGCGCATGTGCCGGCACCTAGCC |
| 2201 | CGCAAGGCCCTGGTGGCCTCCAGGGTGCCTCCACCTGA |

In certain embodiments, the nucleic acid molecule encoding ABCD1 is codon optimized and/or codon modified from the wild-type nucleotide sequence (e.g., encode the same amino acid sequence but with different codon usage). An example of a nucleotide sequence encoding human ABCD1 with a silent mutations is (SEQ ID NO: 4):

| | |
|---|---|
| 1 | ATGCCGGTGCTCTCCAGGCCCCGGCCCTGGCGGGGGAACACGCTGAAGCG |
| 51 | CACGGCCGTGCTCCTGGCCCTCGCGGCCTATGGAGCCCACAAAGTCTACC |
| 101 | CCTTGGTGCGCCAGTGCCTGGCCCCGGCCAGGGGTCTTCAGGCGCCCGCC |
| 151 | GGGGAGCCCACGCAGGAGGCCTCCGGGGTCGCGGCGGCCAAAGCTGGCAT |
| 201 | GAACCGGGTATTCCTGCAGCGGCTCCTGTGGCTCCTGCGGCTGCTGTTCC |
| 251 | CCCGGGTCCTGTGCCGGGAGACGGGGCTGCTGGCCCTGCACTCGGCCGCC |
| 301 | TTGGTGAGCCGCACCTTCCTGTCGGTGTATGTGGCCCGCCTGGACGGAAG |
| 351 | GCTGGCCCGCTGCATCGTCCGCAAGGACCCGCGGGCTTTTGGCTGGCAGC |
| 401 | TGCTGCAGTGGCTCCTCATCGCCCTCCCTGCTACCTTCGTCAACAGTGCC |
| 451 | ATCCGTTACCTGGAGGGCCAACTGGCCCTGTCGTTCCGCAGCCGTCTGGT |
| 501 | GGCCCACGCCTACCGCCTCTACTTCTCCCAGCAGACCTACTACCGGGTCA |
| 551 | GCAACATGGACGGGCGGCTTCGCAACCCTGACCAGTCTCTGACGGAGGAC |
| 601 | GTGGTGGCCTTTGCGGCCTCTGTGGCCCACCTCTACTCCAACCTGACCAA |
| 651 | GCCACTCCTGGACGTGGCTGTGACTTCCTACACCCTGCTTCGGGCGGCCC |
| 701 | GCTCCCGTGGAGCCGGCACAGCCTGGCCCTCGGCCATCGCCGGCCTCGTG |
| 751 | GTGTTCCTCACGGCCAACGTGCTGCGGGCCTTCTCGCCCAAGTTCGGGGA |
| 801 | GCTGGTGGCAGAGGAGGCGCGGCGGAAGGGGGAGCTGCGCTACATGCACT |
| 851 | CGCGTGTGGTGGCCAACTCGGAGGAGATCGCCTTCTATGGGGGCCATGAG |
| 901 | GTGGAGCTGGCCCTGCTACAGCGCTCCTACCAGGACCTGGCCTCGCAGAT |
| 951 | CAACCTCATCCTTCTGGAACGCCTGTGGTATGTTATGCTGGAGCAGTTCC |
| 1001 | TCATGAAGTATGTGTGGAGCGCCTCGGGCCTGCTCATGGTGGCTGTCCCC |
| 1051 | ATCATCACTGCCACTGGCTACTCAGAGTCAGATGCAGAGGCCGTGAAGAA |
| 1101 | GGCAGCCTTGGAAAAGAAGGAGGAGGAGCTGGTGAGCGAGCGCACAGAAG |
| 1151 | CCTTCACTATTGCCCGCAACCTCCTGACAGCGGCTGCAGATGCCATTGAG |
| 1201 | CGGATCATGTCGTCGTACAAGGAGGTGACGGAGCTGGCTGGCTACACAGC |
| 1251 | CCGGGTGCACGAGATGTTCCAGGTATTTGAAGATGTTCAGCGCTGTCACT |
| 1301 | TCAAGAGGCCCAGGGAGCTAGAGGACGCTCAGGCGGGGTCTGGGACCATA |
| 1351 | GGCCGGTCTGGTGTCCGTGTGGAGGGCCCCCTGAAGATCCGAGGCCAGGT |
| 1401 | GGTGGATGTGGAACAGGGGATCATCTGCGAGAACATCCCCATCGTCACGC |
| 1451 | CCTCAGGAGAGGTGGTGGTGGCCAGCCTCAACATCAGGGTGGAGGAAGGC |
| 1501 | ATGCATCTGCTCATCACAGGCCCCAATGGCTGCGGCAAGAGCTCCCTGTT |
| 1551 | CCGGATCCTGGGTGGGCTCTGGCCCACGTACGGTGGTGTGCTCTACAAGC |
| 1601 | CCCCACCCCAGCGCATGTTCTACATCCCGCAGAGGCCCTACATGTCTGTG |
| 1651 | GGCTCCCTGCGTGACCAGGTGATCTACCCGGACTCAGTGGAGGACATGCA |
| 1701 | AAGGAAGGGCTACTCGGAGCAGGACCTGGAAGCCATCCTGGACGTCGTGC |
| 1751 | ACCTGCACCACATCCTGCAGCGGGAGGGAGGTTGGGAGGCTATGTGTGAC |
| 1801 | TGGAAGGACGTCCTGTCGGGTGGCGAGAAGCAGAGAATCGGCATGGCCCG |
| 1851 | CATGTTCTACCACAGGCCCAAGTACGCCCTCCTGGATGAATGCACCAGCG |
| 1901 | CCGTGAGCATCGACGTGGAAGGCAAGATCTTCCAGGCGGCCAAGGACGCG |
| 1951 | GGCATTGCCCTGCTCTCCATCACCCACCGGCCCTCCCTGTGGAAATACCA |
| 2001 | CACACACTTGCTACAGTTCGATGGGGAGGGCGGCTGGAAGTTCGAGAAGC |
| 2051 | TGGACTCAGCTGCCCGCCTGAGCCTGACGGAGGAGAAGCAGCGGCTGGAG |
| 2101 | CAGCAGCTGGCGGGCATTCCCAAGATGCAGCGGCGCCTCCAGGAGCTCTG |
| 2151 | CCAGATCCTGGGCGAGGCCGTGGCCCCAGCGCATGTGCCGGCACCTAGCC |
| 2201 | CGCAAGGCCCTGGTGGCCTCCAGGGTGCCTCCACCTGA |

In certain embodiment, the amino acid (e.g., encoded by the above nucleotide sequences) or nucleotide sequence of ABCD1 has at least 80, 81, 82, 83, 84, 85, 86, 87, 88, 89, 90, 91, 92, 93, 94, 95, 96, 97, 98, or 99% identity with any of the above sequences.

Third, the Woodchuck Post-Regulatory Element, or WPRE can be placed outside the integrating sequence to increase the safety of the vector. In a particular embodiment, the WPRE is 3′ of the 3′LTR. The WPRE increases the titer of the lentivirus, but it can undergo chromosomal rearrangement upon integration. In order to preserve the ability of WPRE to increase viral titers without having this viral element in the integrating sequence, the WPRE can be removed from the integrating portion and added, for example, after the 3′LTR. In addition, a polyadenylation signal (e.g., a bovine growth hormone polyA tail) can be inserted after the WPRE region to increase lentiviral titers (Zaiss, et al. (2002) J. Virol., 76 (14): 7209-19). In certain embodiments, the WPRE is the Woodchuck Hepatitis Virus Posttranscriptional Regulatory Element. In certain embodiments, the WPRE comprises (SEQ ID NO: 5):

GTCGACAATCA ACCTCTGGAT TACAAAATTT
GTGAAAGATT GACTGGTATT CTTAACTATG TTGCTCCTTT TACGCTATGT GGATACGCTG
CTTTAATGCC TTTGTATCAT GCTATTGCTT CCCGTATGGC TTTCATTTTC TCCTCCTTGT
ATAAATCCTG GTTGCTGTCT CTTTATGAGG AGTTGTGGCC CGTTGTCAGG CAACGTGGCG
TGGTGTGCAC TGTGTTTGCT GACGCAACCC CCACTGGTTG GGGCATTGCC ACCACCTGTC
AGCTCCTTTC CGGGACTTTC GCTTTCCCCC TCCCTATTGC CACGGCGGAA CTCATCGCCG
CCTGCCTTGC CCGCTGCTGG ACAGGGGCTC GGCTGTTGGG CACTGACAAT TCCGTGGTGT
TGTCGGGGAA GCTGACGTCC TTTCCATGGC TGCTCGCCTG TGTTGCCACC TGGATTCTGC
GCGGGACGTC CTTCTGCTAC GTCCCTTCGG CCCTCAATCC AGCGGACCTT CCTTCCCGCG
GCCTGCTGCC GGCTCTGCGG CCTCTTCCGC GTCTTCGCCT TCGCCCTCAG ACGAGTCGGA
TCTCCCTTTG GGCCGCCTCC CCGCCTGGAA TTCGAGCTCG GTACC,

or the complement thereof. In certain embodiments, the nucleotide sequence of the WPRE comprises the nucleotide sequence provided in FIG. 12. In certain embodiments, the nucleotide sequence of the WPRE has at least 80, 81, 82, 83, 84, 85, 86, 87, 88, 89, 90, 91, 92, 93, 94, 95, 96, 97, 98, or 99% identity with any of the above sequences.

Fourth, in certain embodiments of the instant invention, the vector may comprise insulators to maximize therapeutic protein expression at a random site of integration and to protect the host genome from possible genotoxicity. Insulators can shelter the transgenic cassette from the silencing effect of non-permissive chromatin sites and, at the same time, protect the genomic environment from the enhancer effect mediated by active regulatory elements (like the LCR) introduced with the vector. The 1.2 Kb cHS4 insulator has been used to rescue the phenotype of thalassemic CD34+BM-derived cells (Puthenveetil, et al. (2004) Blood, 104 (12): 3445-53). Further, fetal hemoglobin can be synthesized in human CD34⁺-derived cells after treatment with a lentiviral vector encoding the gamma-globin gene, either in association with the 400 bp core of the cHS4 insulator or with a lentiviral vector carrying an shRNA targeting the gamma-globin gene repressor protein BCL 11A (Wilber, et al. (2011) Blood, 117 (10): 2817-26). The HS2 enhancer of the GATA1 gene has also been used to achieve high beta-globin gene expression in human cells from patients with beta-thalassemia (Miccio, et al. (2011) PLOS One, 6 (12): e27955). The use of a 200 bp insulator, derived from the promoter of the ankyrin gene, resulted in a significant amelioration of the thalassemic phenotype in mice and high level of expression was reached in both human thalassemic and SCD cells (Breda, et al. (2012) PloS one 7 (3): e32345). In certain embodiments, the ankyrin insulator comprises (SEQ ID NO: 6):

gtgc gggccaggcc cccgagggcc ttatcggccc cagaggcgct
tgctgtcggg ccgggcgctc ccggcacggg cgggcggagg
ggtggcgccc gcctggggac cgcagattac aagagcacct
cctcccccaa ccccaggagg ccccgctccc caggcctcgg
ccggcgcgga cccctggttg ccccgg,

or the complement thereof. In certain embodiments, the ankyrin insulator comprises the nucleotide sequence provided in FIG. 12. The foamy virus has a 36-bp insulator located in its long terminal repeat (LTR) which reduces its genotoxic potential (Goodman, et al. (2018) J. Virol., 92: e01639-17). In certain embodiments, the foamy virus insulator comprises

AAGGGAGACATCTAGTGATATAAGTGTGAACTACAC (SEQ ID NO: 7) or the complement thereof. In certain embodiments, the foamy virus insulator comprises the sequence provided in FIG. 12. In certain embodiments, the insulator sequence has at least 80, 81, 82, 83, 84, 85, 86, 87, 88, 89, 90, 91, 92, 93, 94, 95, 96, 97, 98, or 99% identity with any of the above sequence.

Fifth, in certain embodiments of the instant invention, the vector comprises a nucleic acid molecule encoding FGE. In certain embodiments, the FGE replaces the nucleic acid molecule encoding the sulfatase. In certain embodiments, the FGE is in addition to the nucleic acid molecule encoding the sulfatase. The nucleic acid molecule encoding FGE may be under the control of the same promoter as the nucleic acid molecule encoding the sulfatase (e.g., separated by an IRES) or the nucleic acid molecule encoding FGE may have its own promoter (e.g., EF1-alpha promoter). In certain embodiments, the FGE may be contained in a nucleic acid or vector separate from the nucleic acid or vector containing the sulfatase. In certain embodiments, the FGE and sulfatase are administered to the cell or subject at the same time (e.g., in the same nucleic acid molecule or vector) or administered at different times (e.g., as part of separate nucleic acids or vectors).

In certain embodiments, the FGE is human. Examples of amino acid and nucleotide sequences of FGE (SUMF1) are provided in GenBank Gene ID: 285362 and GenBank Accession Nos. NM_001164674.2, NP_001158146.1, NM_001164675.2, NP_001158147.1, NM_182760.4 and NP_877437.2. In certain embodiments, the FGE is isoform 1. An example of an amino acid sequence of human FGE is (SEQ ID NO: 8):

| | |
|---|---|
| 1 | MAAPALGLVC GRCPELGLVL LLLLLSLLCG AAGSQEAGTG AGAGSLAGSC GCGTPQRPGA |
| 61 | HGSSAAAHRY SREANAPGPV PGERQLAHSK MVPIPAGVFT MGTDDPQIKQ DGEAPARRVT |
| 121 | IDAFYMDAYE VSNTEFEKFV NSTGYLTEAE KFGDSFVFEG MLSEQVKTNI QQAVAAAPWW |
| 181 | LPVKGANWRH PEGPDSTILH RPDHPVLHVS WNDAVAYCTW AGKRLPTEAE WEYSCRGGLH |
| 241 | NRLFPWGNKL QPKGQHYANI WQGEFPVTNT GEDGFQGTAP VDAFPPNGYG LYNIVGNAWE |
| 301 | WTSDWWTVHH SVEETLNPKG PPSGKDRVKK GGSYMCHRSY CYRYRCAARS QNTPDSSASN |
| 361 | LGFRCAADRL PTMD |

Amino acids 1-33 are a signal peptide and amino acids 34-374 is the mature protein. The FGE of the instant invention may contain the signal peptide or it may be absent. An example of a nucleotide sequence encoding human FGE is (SEQ ID NO: 9):

| | |
|---|---|
| 1 | gggtcacatg gcccgcggga caacatggct gcgcccgcac tagggctggt gtgtggacgt |
| 61 | tgccctgagc tgggtctcgt cctcttgctg ctgctgctct cgctgctgtg tggagcggca |
| 121 | gggagccagg aggccgggac cggtgcgggc gcggggtccc ttgcgggttc ttgcggctgc |
| 181 | ggcacgcccc agcggcctgg cgcccatggc agttcggcag ccgctcaccg atactcgcgg |
| 241 | gaggctaacg ctccgggccc cgtacccgga gagcggcaac tcgcgcactc aaagatggtc |
| 301 | cccatccctg ctggagtatt tacaatgggc acagatgatc ctcagataaa gcaggatggg |
| 361 | gaagcacctg cgaggagagt tactattgat gccttttaca tggatgccta tgaagtcagt |
| 421 | aatactgaat ttgagaagtt tgtgaactca actggctatt tgacagaggc tgagaagttt |
| 481 | ggcgactcct ttgtctttga aggcatgttg agtgagcaag tgaagaccaa tattcaacag |
| 541 | gcagttgcag ctgctccctg gtggttacct gtgaaaggcg ctaactggag acacccagaa |
| 601 | gggcctgact ctactattct gcacaggccg gatcatccag ttctccatgt gtcctggaat |
| 661 | gatgcggttg cctactgcac ttgggcaggg aagcggctgc ccacggaagc tgagtgggaa |
| 721 | tacagctgtc gaggaggcct gcataataga cttttcccct ggggcaacaa actgcagccc |
| 781 | aaaggccagc attatgccaa catttggcag ggcgagtttc cggtgaccaa cactggtgag |
| 841 | gatggcttcc aaggaactgc gcctgttgat gccttccctc ccaatggtta tggcttatac |
| 901 | aacatagtgg ggaacgcatg ggaatggact tcagactggt ggactgttca tcattctgtt |
| 961 | gaagaaacgc ttaacccaaa aggtccccct tctgggaaag accgagtgaa gaaaggtgga |
| 1021 | tcctacatgt gccataggtc ttattgttac aggtatcgct gtgctgctcg gagccagaac |
| 1081 | acacctgata gctctgcttc gaatctggga ttccgctgtg cagccgaccg cctgcccact |
| 1141 | atggactgac aaccaaggaa agtcttcccc agtccaagga gcagtcgtgt ctgacctaca |
| 1201 | ttgggctttt ctcagaactt tgaacgatcc catgcaaaga attcccaccc tgaggtgggt |
| 1261 | tacatacctg cccaatggcc aaaggaaccg ccttgtgaga ccaaattgct gacctgggtc |
| 1321 | agtgcatgtg ctttatggtg tggtgcatct ttggagatca tcaccatatt ttacttttga |
| 1381 | gagtctttaa agaggaaggg gagtggaggg aaccctgagc taggcttcag gaggcccgcg |
| 1441 | tcctacgcag gctctgccac aggggttaga ccccaggtcc gacgcttgac cttcctgggc |
| 1501 | ctcaagtgcc ctcccctatc aaatgaaggg atggacagca tgacctctgg gtgtctctcc |
| 1561 | aactcaccag ttctaaaaag ggtatcagat tctattgtga cttcatagtg agaatttatt |
| 1621 | atagattatt ttttagctat tttttccatg tgtgaacctt gagtgatact aatcatgtaa |
| 1681 | agtaagagtt ctcttatgta ttattttcgg aagaggggtg tggtgactcc tttatattcg |
| 1741 | tactgcactt tgtttttcca aggaaatcag tgtcttttac gttgttatga tgaatcccac |
| 1801 | atggggccgg tgatggtatg ctgaagttca gccgttgaac acataggaat gtctgtgggg |
| 1861 | tgactctact gtgctttatc ttttaacatt aagtgccttt ggttcagagg ggcagtcata |
| 1921 | agctctgttt ccccctctcc ccaaagcctt cagcgaacgt gaaatgtgcg ctaaacgggg |
| 1981 | aaacctgttt aattctagat atagggaaaa aggaacgagg accttgaatg agctatattc |
| 2041 | agggtatccg gtattttgta atagggaata ggaaaccttg ttggctgtgg aatatccgat |
| 2101 | gctttgaatc atgcactgtg ttgaataaac gtatctgcta aatcagg |

Nucleotides 25-1149 translate into the above amino acid sequence and also encodes for FGE. In certain embodiments, the nucleotide sequence encoding human FGE comprises nucleotides 25-1149.

In certain embodiments, the nucleic acid molecule encoding FGE is codon optimized and/or codon modified from the wild-type nucleotide sequence (e.g., encode the same amino acid sequence but with different codon usage). In certain embodiment, the nucleotide sequence of FGE comprises the sequence provided in FIG. 11. In certain embodiment, the amino acid or nucleotide sequence of FGE has at least 80, 81, 82, 83, 84, 85, 86, 87, 88, 89, 90, 91, 92, 93, 94, 95, 96, 97, 98, or 99% identity with any of the above sequences.

Sixth, in certain embodiments of the instant invention, the promoter is the EF1-alpha promoter. In certain embodiments, the EF1-alpha promoter is human. In certain embodiments, the nucleotide sequence of the EF1-alpha promoter comprises the nucleotide sequence provided in FIG. 11 or 12. In certain embodiment, the nucleotide sequence of the PGK promoter is from GenBank Accession No. J04617.1. In certain embodiments, the nucleotide sequence of the EF1-alpha promoter comprises (SEQ ID NO: 10):

GGCTCCGGTGCCCGTCAGTGGGCAGAGCGCACATCGCCCACAGTCCCCG
AGAAGTTGGGGGGAGGGGTCGGCAATTGAACCGGTGCCTAGAGAAGGTG
GCGCGGGGTAAACTGGGAAAGTGATGTCGTGTACTGGCTCCGCCTTTTT
CCCGAGGGTGGGGGAGAACCGTATATAAGTGCAGTAGTCGCCGTGAACG
TTCTTTTTCGCAACGGGTTTGCCGCCAGAACACAGGTAAGTGCCGTGTG
TGGTTCCCGCGGGCCTGGCCTCTTTACGGGTTATGGCCCTTGCGTGCCT
TGAATTACTTCCACGCCCCTGGCTGCAGTACGTGATTCTTGATCCCGAG
CTTCGGGTTGGAAGTGGGTGGGAGAGTTCGAGGCCTTGCGCTTAAGGAG
CCCCTTCGCCTCGTGCTTGAGTTGAGGCCTGGCCTGGGCGCTGGGGCCG
CCGCGTGCGAATCTGGTGGCACCTTCGCGCCTGTCTCGCTGCTTTCGAT
AAGTCTCTAGCCATTTAAAATTTTTGATGACCTGCTGCGACGCTTTTTT
TCTGGCAAGATAGTCTTGTAAATGCGGGCCAAGATCTGCACACTGGTAT
TTCGGTTTTTGGGGCCGCGGGCGGCGACGGGGCCCGTGCGTCCCAGCGC
ACATGTTCGGCGAGGCGGGGCCTGCGAGCGCGGCCACCGAGAATCGGAC
GGGGGTAGTCTCAAGCTGGCCGGCCTGCTCTGGTGCCTGGCCTCGCGCC
GCCGTGTATCGCCCCGCCCTGGGCGGCAAGGCTGGCCCGGTCGGCACCA
GTTGCGTGAGCGGAAAGATGGCCGCTTCCCGGCCCTGCTGCAGGGAGCT
CAAAATGGAGGACGCGGCGCTCGGGAGAGCGGGCGGGTGAGTCACCCAC
ACAAAGGAAAAGGGCCTTTCCGTCCTCAGCCGTCGCTTCATGTGACTCC
ACGGAGTACCGGGCGCCGTCCAGGCACCTCGATTAGTTCTCGAGCTTTT
GGAGTACGTCGTCTTTAGGTTGGGGGGAGGGGTTTTATGCGATGGAGTT
TCCCCACACTGAGTGGGTGGAGACTGAAGTTAGGCCAGCTTGGCACTTG
ATGTAATTCTCCTTGGAATTTGCCCTTTTTGAGTTTGGATCTTGGTTCA
TTCTCAAGCCTCAGACAGTGGTTCAAAGTTTTTTTCTTCCATTTCAGGT
GTCGTGAg.

In certain embodiments, the nucleotide sequence of the EF1-alpha promoter has at least 80, 81, 82, 83, 84, 85, 86, 87, 88, 89, 90, 91, 92, 93, 94, 95, 96, 97, 98, or 99% identity with the above sequence.

Seventh, while the invention is generally described with the EF1-alpha promoter, the promoter may be replaced with the CD68 promoter. In certain embodiments of the instant invention, the promoter is the CD68 promoter. In certain embodiments, the CD68 promoter is human. In certain embodiments, the nucleotide sequence of the CD68 comprises the nucleotide sequence provided in FIG. 11. In certain embodiment, the nucleotide sequence of the CD68 promoter is from GenBank 20 GeneID 968. In certain embodiments, the nucleotide sequence of the CD68 promoter comprises (SEO MD NO: 11)

gtacaggggtcagcccagaggtccaggggaaaggagtggaaaccgattt
ccccaccaagggaggggcctgtacctcagctgttcccatagctacttgc
cacaactgccaagcaagtttcgctgagtttgacacatggatccctgtgg
atcaactgccctaggactccgtttgcacccatgtgacactgttgacttt
gccctgatgaagcagggccaacagtcccctaacttaattacaaaaacta
atgactaagagagaggtggctagagctgaggcccctgagtcaggctgtg
ggtgggatcatctccagtacaggaagtgagactttcatttcctcctttc
caagagagggctgagggagcagggttgagcaactggtgcagacagccta
gctggactttgggtgaggcggttcagcc.

In certain embodiments, the nucleotide sequence of the CD68 promoter has at least 80, 81, 82, 83, 84, 85, 86, 87, 88, 89, 90, 91, 92, 93, 94, 95, 96, 97, 98, or 99% identity with the above sequence.

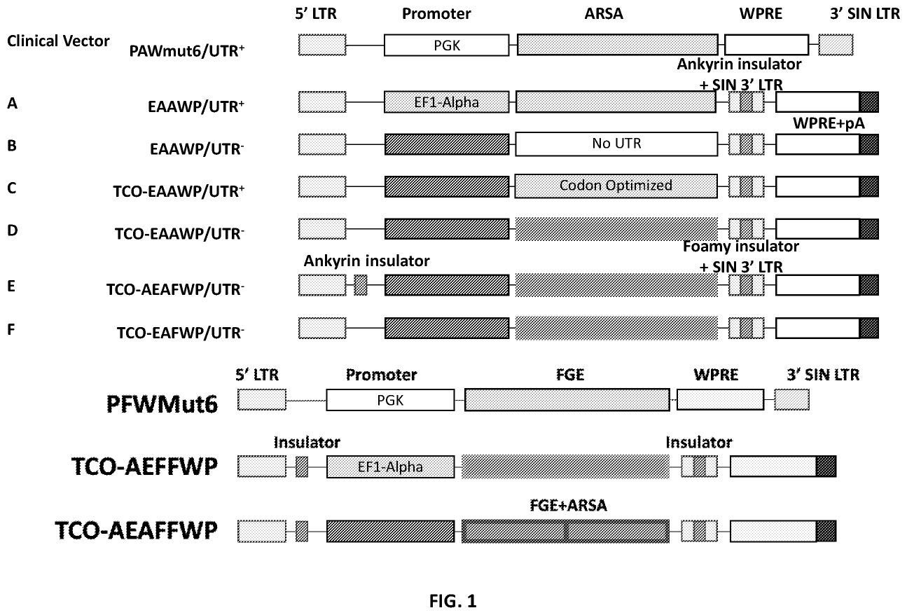

In certain embodiment, the nucleic acid or viral vector of the instant invention has a nucleotide sequence identical to those presented herein or they can have least 80, 81, 82, 83, 84, 85, 86, 87, 88, 89, 90, 91, 92, 93, 94, 95, 96, 97, 98, or 99% identity to the nucleotide sequence of a viral vector disclosed herein or to an element of a nucleotide sequence of a viral vector disclosed herein (e.g., the sequences provided herein (e.g., FIGS. 11 and 12) and in U.S. Patent Application Publication 2018/0008725; WO 2019/213011; and WO 2020/264488). In a particular embodiment, the nucleic acid or viral vector of the instant invention comprises the elements of any vector set forth in FIG. 1, particularly in the orientation presented.

The present disclosure provides compositions and methods for the inhibition, prevention, and/or treatment of a disease or disorder such as multiple sulfatase deficiency (MSD), metachromatic leukodystrophy (MLD) and/or a sulfatase deficiency. In certain embodiments, the sulfatase deficiency is a deficiency of a lysosomal sulfatase. In certain embodiments, the sulfatase deficiency presents as a lysosomal storage disease. In certain embodiments, the disease or disorder is ALD. In certain embodiments, the disease or disorder is MPS IIIA. In certain embodiments, the disease or disorder is Krabbe. In certain embodiments, the disease or disorder is ALS.

In accordance with another aspect of the instant invention, methods of transducing cells with a nucleic acid or viral vector of the instant invention are provided. In a particular embodiment, the transduction is performed with the adjuvant/enhancer LentiBoost™ or cyclosporine H. In a particular embodiment, the viral vector is pseudotyped with Cocal envelope. In a particular embodiment, the viral vector is pseudotyped with VSV-G envelope.

In accordance with the instant invention, compositions and methods are provided for increasing protein (e.g., therapeutic protein) production such as sulfatase (e.g., ARSA) and/or FGE production in a cell or subject. The method comprises administering a nucleic acid or viral vector of the instant invention to the cell, particularly a hematopoietic stem cell, bone marrow cell, microglia, macrophage and/or fibroblast, or subject. In a particular embodiment, the subject has a disease or disorder such as multiple sulfatase deficiency (MSD), leukodystrophy (e.g., metachromatic leukodystrophy (MLD)) and/or a sulfatase deficiency. In a particular embodiment, the subject has multiple sulfatase deficiency (MSD) and the nucleic acid or viral vector administered to the subject encodes FGE, optionally with ARSA. In a particular embodiment, the subject has metachromatic leukodystrophy (MLD) and the nucleic acid or viral vector administered to the subject encodes ARSA and, optionally, FGE. In a particular embodiment, the subject has metachromatic leukodystrophy (MLD) and the nucleic acid or viral vector administered to the subject encodes ARSA and FGE. In a particular embodiment, the subject has a sulfatase deficiency and the nucleic acid or viral vector administered to the subject encodes the deficient sulfatase and, optionally, FGE. In a particular embodiment, the subject has ALD and the nucleic acid or viral vector administered to the subject encodes ABCD1. In a particular embodiment, the subject has MPS IIIA and the nucleic acid or viral vector administered to the subject encodes SGSH. In a particular embodiment, the subject has Krabble and the nucleic acid or viral vector administered to the subject encodes GALC and, optionally, ARSA. In a particular embodiment, the subject has ALS and the nucleic acid or viral vector administered to the subject encodes SOD1. The viral vector may be administered in a composition further comprising at least one pharmaceutically acceptable carrier.

In accordance with another aspect of the instant invention, compositions and methods for inhibiting (e.g., reducing or slowing), treating, and/or preventing a disease or disorder (e.g., multiple sulfatase deficiency (MSD), leukodystrophy (e.g., metachromatic leukodystrophy (MLD)), and/or a sulfatase deficiency). In a particular embodiment, the methods comprise administering to a subject in need thereof a nucleic acid or viral vector of the instant invention. The viral vector may be administered in a composition further comprising at least one pharmaceutically acceptable carrier. The nucleic acid or viral vector may be administered via an ex vivo methods wherein the nucleic acid or viral vector is delivered to a hematopoietic stem cell, bone marrow cell, microglia, macrophage, and/or fibroblasts, particularly autologous ones, and then the cells are administered to the subject. In a particular embodiment, the method comprises isolating hematopoietic cells, bone marrow cells, microglia, macrophage, and/or fibroblasts from a subject, delivering a nucleic acid or viral vector of the instant invention to the cells, and administering the treated cells to the subject. The methods of the instant invention may further comprise monitoring the disease or disorder in the subject after administration of the composition(s) of the instant invention to monitor the efficacy of the method. For example, the subject may be monitored for characteristics of sulfate deficiency.

The methods of the instant invention may further comprise the administration of another therapeutic regimen. For example, the methods may further comprise administering another therapeutic or therapy for treatment of the disease or disorder (e.g., multiple sulfatase deficiency (MSD), metachromatic leukodystrophy (MLD) and/or a sulfatase deficiency) or symptoms thereof.

As explained hereinabove, the compositions of the instant invention are useful for increasing protein production and treating a disease or disorder or for increasing sulfate production and for treating multiple sulfatase deficiency (MSD), metachromatic leukodystrophy (MLD) and/or a sulfatase deficiency. A therapeutically effective amount of the composition may be administered to a subject in need thereof. The dosages, methods, and times of administration are readily determinable by persons skilled in the art, given the teachings provided herein.

The components as described herein will generally be administered to a patient as a pharmaceutical preparation. The term “patient” or “subject” as used herein refers to human or animal subjects. The components of the instant invention may be employed therapeutically, under the guidance of a physician for the treatment of the indicated disease or disorder.

The pharmaceutical preparation comprising the components of the invention may be conveniently formulated for administration with an acceptable medium (e.g., pharmaceutically acceptable carrier) such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the agents in the chosen medium may be varied and the medium may be chosen based on the desired route of administration of the pharmaceutical preparation. Except insofar as any conventional media or agent is incompatible with the agents to be administered, its use in the pharmaceutical preparation is contemplated.

The compositions of the present invention can be administered by any suitable route, for example, by injection (e.g., for local (direct) or systemic administration), oral, pulmonary, topical, nasal or other modes of administration. The composition may be administered by any suitable means, including parenteral, intramuscular, intravenous, intraarterial, intraperitoneal, subcutaneous, topical, inhalatory, transdermal, intrapulmonary, intraareterial, intrarectal, intramuscular, and intranasal administration. In a particular embodiment, the composition is administered directly to the blood stream (e.g., intravenously). In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., polysorbate 80), anti oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington: The Science and Practice of Pharmacy, 21st edition, Philadelphia, PA. Lippincott Williams & Wilkins. The pharmaceutical composition of the present invention can be prepared, for example, in liquid form, or can be in dried powder form (e.g., lyophilized for later reconstitution).

As used herein, “pharmaceutically acceptable carrier” includes any and all solvents, dispersion media and the like which may be appropriate for the desired route of administration of the pharmaceutical preparation, as exemplified in the preceding paragraph. The use of such media for pharmaceutically active substances is known in the art. Except insofar as any conventional media or agent is incompatible with the molecules to be administered, its use in the pharmaceutical preparation is contemplated.

Pharmaceutical compositions containing a compound of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous. Injectable suspensions may be prepared, in which case appropriate liquid carriers, suspending agents and the like may be employed. Pharmaceutical preparations for injection are known in the art. If injection is selected as a method for administering the therapy, steps should be taken to ensure that sufficient amounts of the molecules reach their target cells to exert a biological effect.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art. The appropriate dosage unit for the administration of the molecules of the instant invention may be determined by evaluating the toxicity of the molecules in animal models. Various concentrations of pharmaceutical preparations may be administered to mice with transplanted human tumors, and the minimal and maximal dosages may be determined based on the results of significant reduction of tumor size and side effects as a result of the treatment. Appropriate dosage unit may also be determined by assessing the efficacy of the treatment in combination with other standard therapies.

The pharmaceutical preparation comprising the molecules of the instant invention may be administered at appropriate intervals, for example, at least twice a day or more until the pathological symptoms are reduced or alleviated, after which the dosage may be reduced to a maintenance level. The appropriate interval in a particular case would normally depend on the condition of the patient.

### Definitions

The singular forms “a,” “an,” and “the” include plural referents unless the context clearly dictates otherwise.

The terms “isolated” is not meant to exclude artificial or synthetic mixtures with other compounds or materials, or the presence of impurities that do not interfere with the fundamental activity, and that may be present, for example, due to incomplete purification, or the addition of stabilizers.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxilliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers. Suitable pharmaceutical carriers are described in Remington: The Science and Practice of Pharmacy, (Lippincott, Williams and Wilkins); Liberman, et al., Eds., Pharmaceutical Dosage Forms, Marcel Decker, New York, N.Y.; and Rowe, et al., Eds., Handbook of Pharmaceutical Excipients, Pharmaceutical Pr.

The term “treat” as used herein refers to any type of treatment that imparts a benefit to a patient suffering from a disease or disorder, including improvement in the condition of the patient (e.g., in one or more symptoms), delay in the progression of the condition, etc.

As used herein, the term “prevent” refers to the prophylactic treatment of a subject who is at risk of developing a condition and/or sustaining a disease or disorder, resulting in a decrease in the probability that the subject will develop conditions associated with the disease or disorder.

A “therapeutically effective amount” of a compound or a pharmaceutical composition refers to an amount effective to prevent, inhibit, or treat a particular injury and/or the symptoms thereof. For example, “therapeutically effective amount” may refer to an amount sufficient to modulate the pathology associated with a specific disease or disorder such as multiple sulfatase deficiency (MSD), metachromatic leukodystrophy (MLD) and/or a sulfatase deficiency.

As used herein, the term “subject” refers to an animal, particularly a mammal, particularly a human.

A “vector” is a genetic element, such as a plasmid, cosmid, bacmid, phage or virus, to which another genetic sequence or element (either DNA or RNA) may be attached so as to bring about the replication and/or expression of the attached sequence or element. A vector may be either RNA or DNA and may be single or double stranded. A vector may comprise expression operons or elements such as, without limitation, transcriptional and translational control sequences, such as promoters, enhancers, translational start signals, polyadenylation signals, terminators, and the like, and which facilitate the expression of a polynucleotide or a polypeptide coding sequence in a host cell or organism.

The following example is provided to illustrate various embodiments of the present invention. It is not intended to limit the invention in any way.

### Example

FIG. 1 provides schematics of the vectors of the instant invention. Building upon the clinical ARSA expressing vector (PAWmut6), a different promoter, ARSA gene sequences and viral insulators have been incorporated to optimize ARSA expression and enhance safety in virally transduced cell lines. The human Elongation Factor 1 alpha (EF1-alpha) promoter is a much stronger promoter across a range of different cell lines compared to the human Phosphoglycerate Kinase (PGK) promoter. The different variations of the ARSA gene are with and without the 5′ and 3′ untranslated regions (UTR+ or UTR−) and an additional codon modified version to distinguish from the endogenous ARSA (Traceable Codon Optimization, TCO). An ankyrin and/or foamy insulator have been incorporated to further reduce the possibility of any genotoxicity caused by the vectors. The Woodchuck Hepatitis Virus Posttranscriptional Regulatory Element (WPRE) has been proven to enhance the performance of viral vectors. However, in order to remove the WPRE from the vector integrating sequence, it was placed directly after the 3′-self inactivating LTR (SIN-LTR) with a strong polyA signal (pA) (Breda, et al., Mol. Ther. (2021) 29(4):1625-1638).

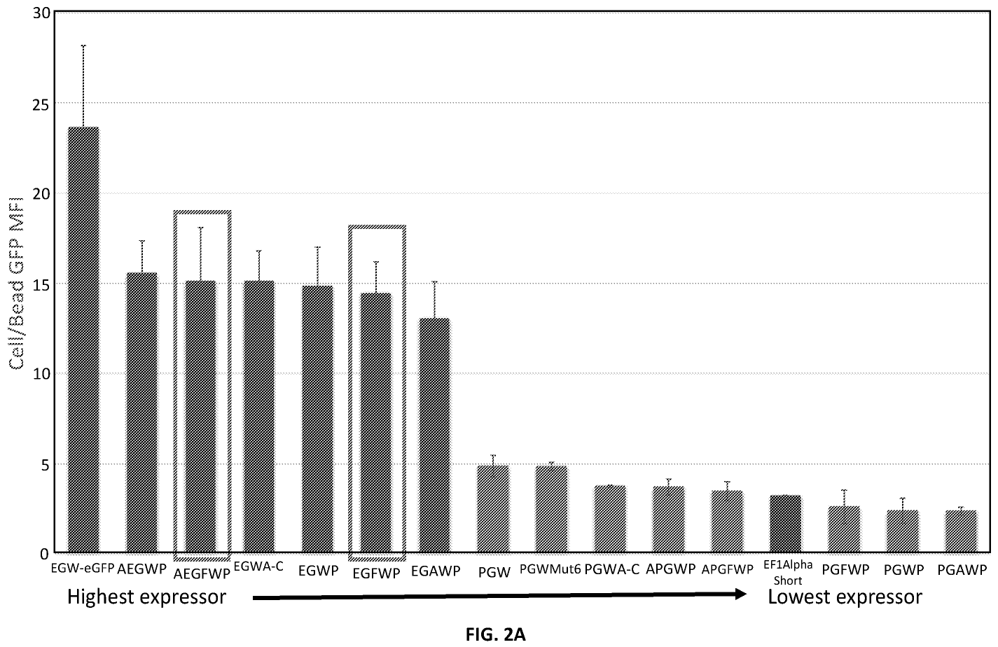

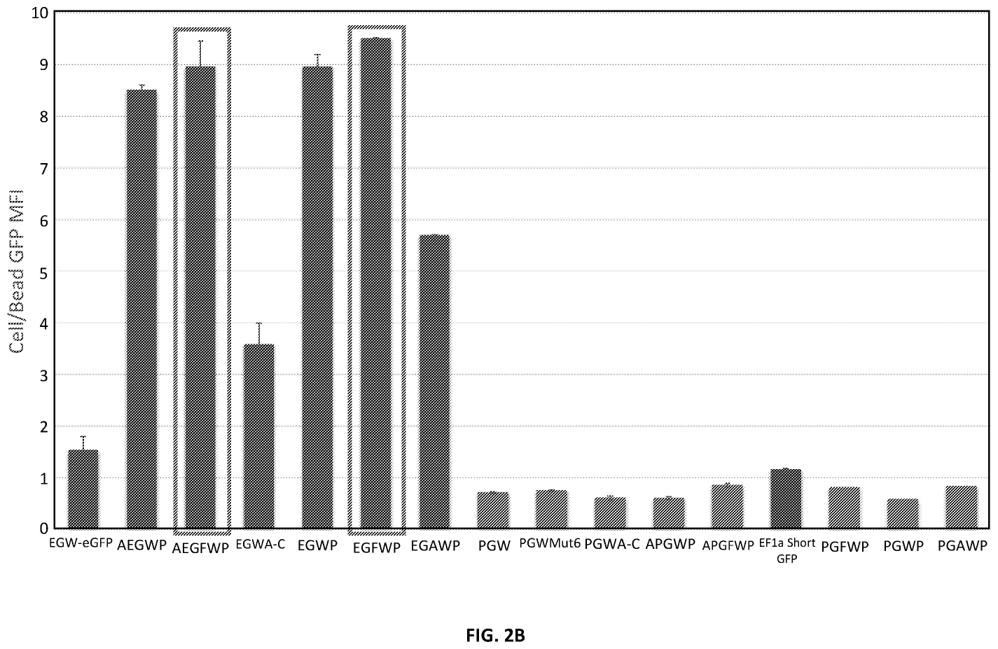

The mean fluorescence index (MFI) of HePG2 cells, a human hepatoma cell line, transduced with different permutations of the EF1-Alpha vector construct was compared to the same constructs with the PGK promotor. EF1-Alpha constructs demonstrated superior GFP fluorescence (FIG. 2A). Outlined vectors have the inclusion of insulator regions for safety and perform optimally in murine erythroleukemia (MEL) cells.

The mean fluorescence index of MEL cells transduced with different permutations of the EF1-Alpha vector construct was compared to the same constructs with the PGK promotor. EF1-Alpha constructs again demonstrated superior GFP fluorescence (FIG. 2B). The observed differences in MEL cells with vectors having an insulator are due to epigenetic silencing in the MEL cells.

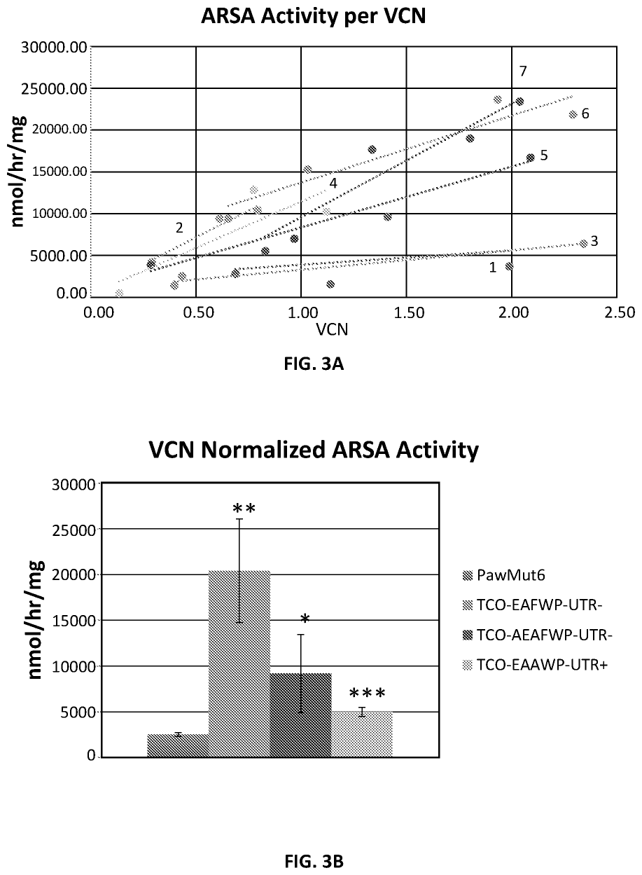

ARSA activity per vector copy number (VCN) was also determined in transduced primary human metachromatic leukodystrophy (MLD) fibroblasts (FIG. 3A). Three vectors, TCO-EAFWP-UTR−, TCO-AEAFWP-UTR− and TCO-EAAWP-UTR+, demonstrated superior activity per VCN in transduced patient MLD fibroblast line GM02331 compared to PawMut6. VCN was quantified by droplet digital PCR (ddPCR) (Breda, et al., Mol. Ther. (2021) 29(4):1625-1638) and enzyme activity was determined according to protocol (Baum, et al., Clin. Chim. Acta (1959) 4(3):453-5). A T test was performed comparing PawMut6 to the three vectors and demonstrated significant difference of expression level (N=3 for each vector) (FIG. 3B). The results were normalized to VCN.

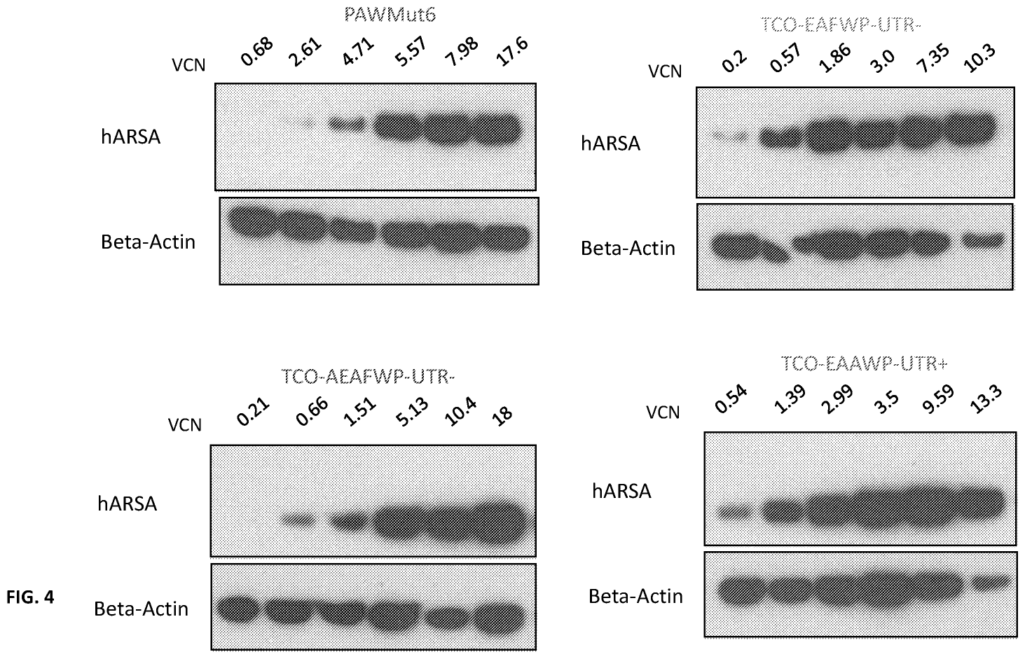

A Western blot analysis of the ARSA vector transduced MLD fibroblasts was also performed (FIG. 4). Specifically, ARSA enzyme assay protein extracts from transduced cells were analyzed by Western blot. As seen in FIG. 4, the extracts demonstrate ARSA protein expression that match enzyme activity levels. Robust amounts of ARSA protein are expressed at modest integration levels of the vectors of the instant invention.

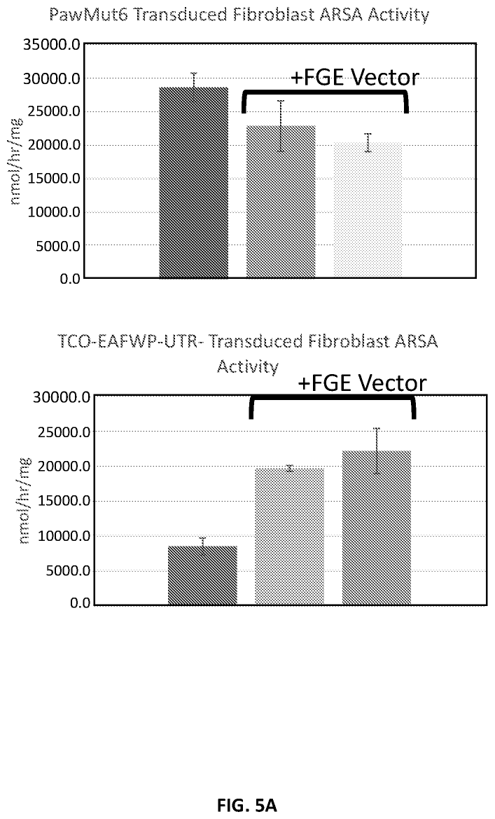

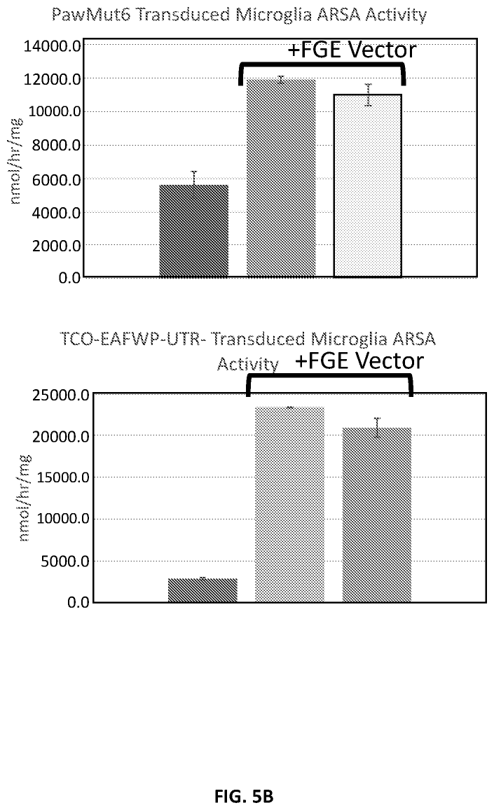

ARSA enzyme requires post-translational modifications and oxidation of a conserved cysteine residue by formylglycine-generating enzyme (FGE) to be active. The activity of ARSA in fibroblasts (FIG. 5A) and microglia (FIG. 5B) was determined before and after FGE transduction. Established fibroblasts and microglia cell lines already transduced with either PawMut6 or TCO-EAFWP-UTR-were assessed for ARSA activity. After transduction with an FGE containing vector, ARSA activity was found to drastically increase, especially in microglia cell lines. The color change of the activity assay can be observed for the microglia cell ARSA activity. This is of importance since FGE is a key activator of ARSA. Therefore, FGE can be used as part of the LV treatment strategy to achieve maximal ARSA activity during overexpression.

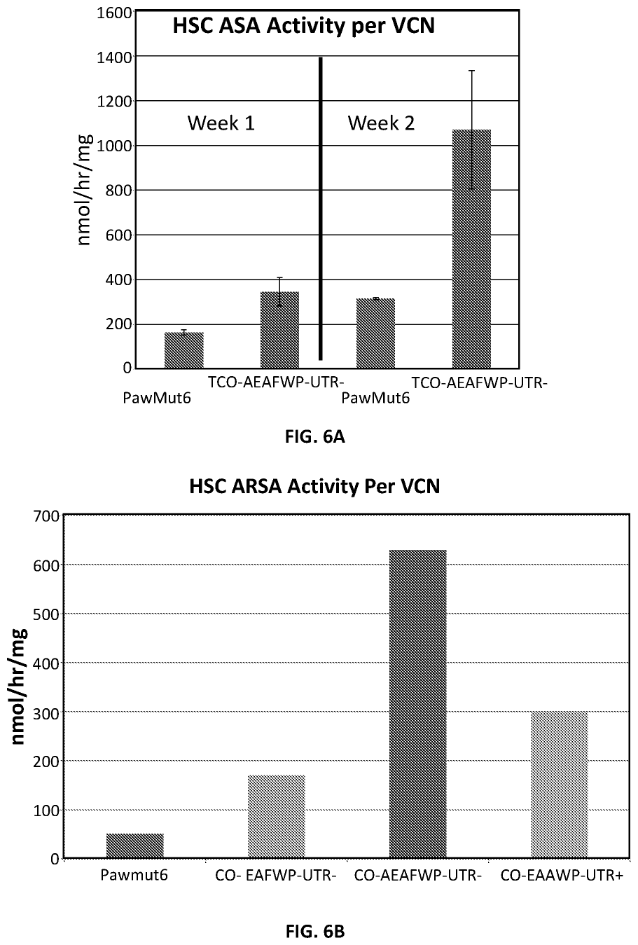

Mouse hematopoietic stem cells (HSC) were transduced with either PawMut6 or TCO-AEAFWP-UTR−. After 2 weeks of transduction ARSA activity demonstrated significantly more activity in TCO-AEAFWP-UTR-transduced HSCs compared to PawMut6 (FIG. 6A). As seen in FIG. 6B, all three vectors (TCO-EAFWP-UTR−, TCO-AEAFWO-UTR− and TCO-EAAWP-UTR+) demonstrate superior activity per VCN in transduced HSCs compared to PawMut6.

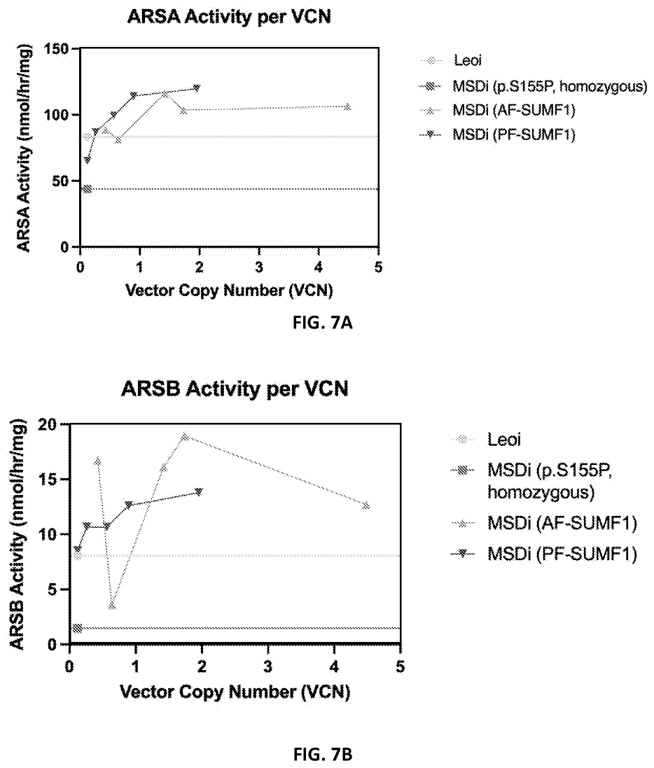

Reconstitution of ARSA activity was also demonstrated in MSD cell lines as VCN of the vector was increased for both a PGK and EF1-alpha FGE variant vectors (FIG. 7A). Recovery of ARSA activity in the disease lines reaches up to and above that of the endogenous activity found in the normal Leoi fibroblast line. Reconstitution of ARSB activity was also demonstrated in MSD cell lines as VCN of the vector was increased for both a PGK and EF1-alpha FGE variant vectors (FIG. 7B). Recovery of ARSB activity in the disease lines reaches up to and above that of the endogenous activity found in the normal Leoi fibroblast line.

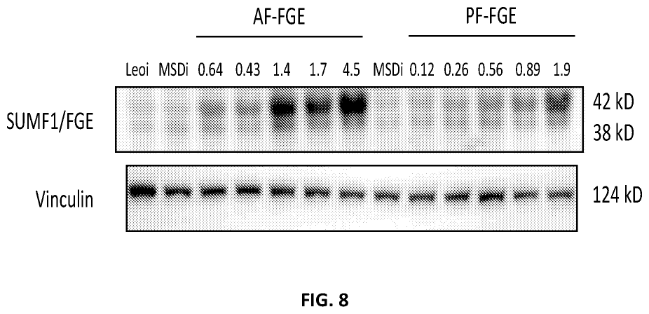

FIG. 8 provides a Western blot analysis of the FGE vector transduced MSD fibroblasts demonstrating SUMF1/FGE expression. Transduced cell extracts demonstrate sulfatase-modifying factor 1 (SUMF1) protein expression increases with increasing VCN and ARSA activity levels. SUMF1 is an essential factor for sulfatase activities and mutations in the SUMF1 gene cause MSD (multiple sulfatase deficiency), an autosomal recessive disease in which the activities of all sulfatases are profoundly reduced. FGE is encoded by the sulfatase-modifying factor 1 gene (SUMF1).

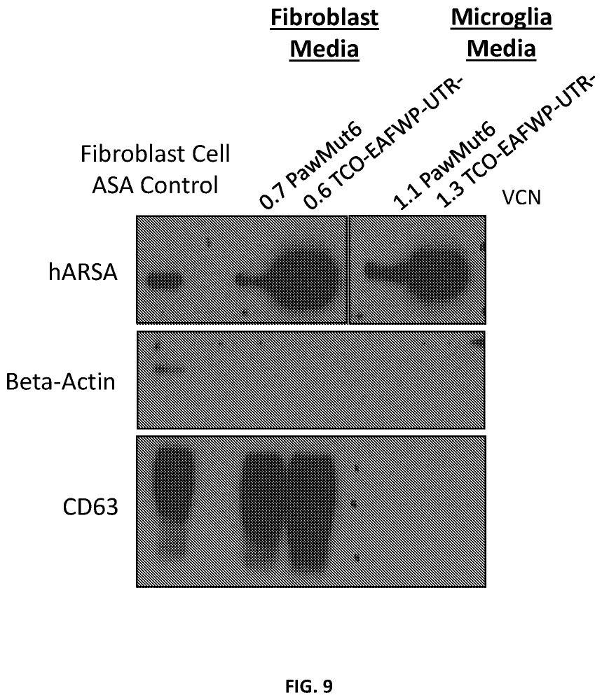

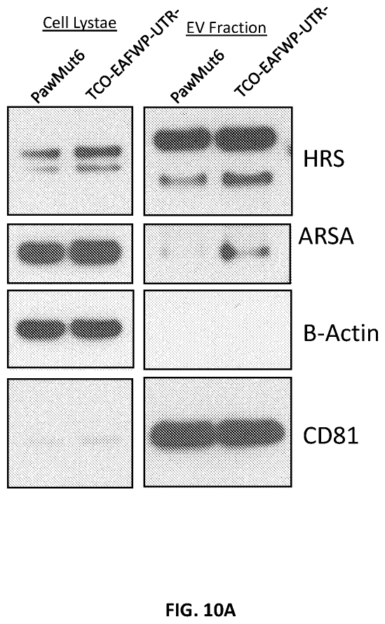

ARSA protein expression was measured from media concentrate of PawMut6 or TCO-EAFWP-UTR-transduced human primary MLD fibroblasts and microglia ARSA-KO cells (FIG. 9). A Western blot was conducted on cell secreted media protein concentrated using a standard acetone precipitation protocol. Primary human fibroblasts and ARSA-KO microglia cell lines were previously transduced with either PawMut6 or TCO-EAFWP-UTR−. Samples were chosen with similar VCN for each cell line. Expression of ARSA proved to be significantly higher in the media extracts of TCO-EAFWP-UTR-transduced cells compared to that of PAWmut6 in both cell lines at similar VCN. B-actin expression was not detected in the media extracts, indicating that ARSA was not associated with or derived from cellular extract components. However, CD63, a well-known membrane marker, was present indicating a possible linkage to secretion of the produced ARSA in some form of extracellular vesicles. CD63 is also visible in media from microglia cells, but at a longer exposure.

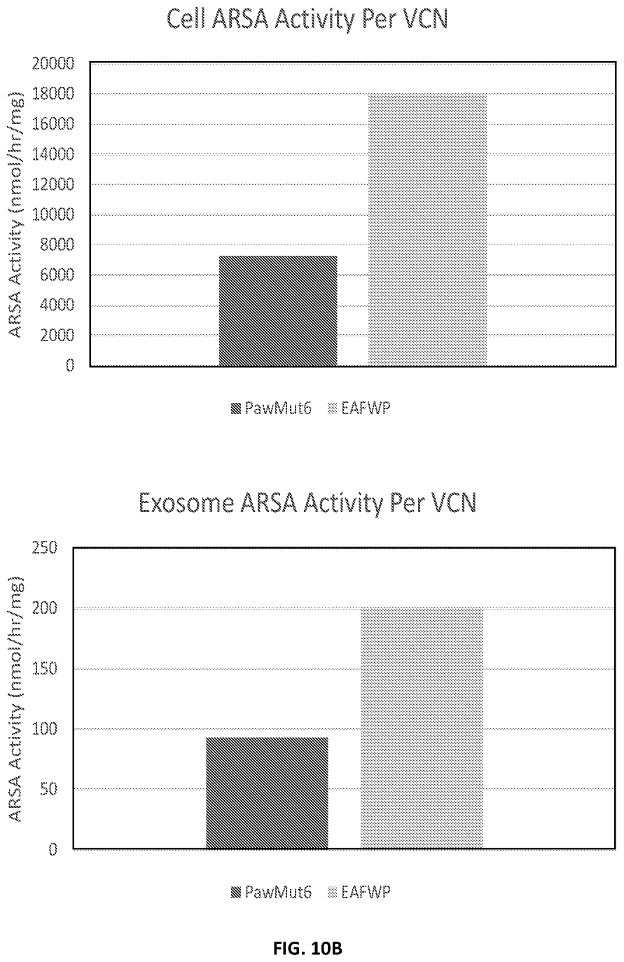

A Western blot was run on a confirmed preparation of extracellular vesicles isolated by ultracentrifugation of media incubated with transduced cells for 72 hours (FIG. 10A). Cell lysate and extracellular vesicle preparations demonstrated higher ARSA expression with TCO-EAFWP-UTR-compared to PawMut6. Other markers for HRS and CD81, which are extracellular vesicle specific, presented a strong signal in the extracellular vesicle fraction from TCO-EAFWP-UTR-transduced cells. ARSA activity assays were also conducted on cell lysate and extracellular vesicle fractions and both demonstrated more activity per VCN for TCO-EAFWP-UTR-compared to PawMut6.

Transplanted irradiated WT mice with GFP donor HCS were transduced with either PawMut6, TCO-EAAWP-UTR+, TCO-AEAFWP-UTR− or TCO-EAFWP-UTR−. The cohort of WT animals transduced with these vectors had bone marrow VCN ranging from 2-12. Mice lived vibrantly post vector transplant and demonstrated no gross hematological abnormalities by flow analysis. Complete blood count (CBC) analysis shows these animals have normal CBC values compared to the control generated animals.

| Bone Marrow | VCN |
|---|---|
| Paw L | 10.6 |
| Paw N | 6.7 |
| Paw R | 6.7 |
| Paw RL | 8.4 |
| Paw R | 8.2 |
| Paw RR | 11.7 |
| AEA RL | 5.9 |
| AEA N | 0.2 |
| AEA RL | 0.9 |
| AEA RR | 1.9 |
| AEA LL | 5.2 |
| EA N | 12 |
| EA R | 2.3 |
| EA RR | 7.3 |
| EA L | 6.2 |
| CO RL | 3.2 |
| CO L | 6.1 |
| CO R | 2.8 |
| CO N | 2.8 |

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1: A lentiviral vector comprising a nucleic acid molecule comprising:
i) a 5′ long terminal repeat (LTR) and a 3′ LTR, wherein at least one of said LTR is self-inactivating;
ii) an Elongation Factor 1 alpha (EF1-alpha) promoter or CD68 promoter;
iii) an insulator element;
iv) a nucleic acid sequence encoding a therapeutic protein;
v) a Woodchuck Post-Regulatory Element (WPRE); and
vi) a polyadenylation signal.

2: The lentiviral vector of claim 1, wherein said therapeutic protein is sulfatase and/or formylglycine-generating enzyme (FGE).

3: The lentiviral vector of claim 2, wherein said sulfatase is arylsulfatase A (ARSA).

4: The lentiviral vector of claim 1, wherein said insulator element is an ankyrin insulator element (Ank) or a foamy virus insulator element.

5: The lentiviral vector of claim 1, wherein said insulator element is within the 3′ LTR.

6: The lentiviral vector of claim 1, wherein said insulator element is between said 5′ LTR and said promoter.

7: The lentiviral vector of claim 1 comprising a nucleic acid sequence encoding a sulfatase and a nucleic acid sequence encoding formylglycine-generating enzyme (FGE).

8: The lentiviral vector of claim 1, wherein said therapeutic protein is selected from the group consisting of ATP binding cassette subfamily D member 1 (ABCD1), N-sulfoglucosamine sulfohydrolase (SGSH), galactocerebrosidase (GALC), and superoxide dismutase 1 gene (SOD1).

9: A cell comprising the lentiviral vector of claim 1.

10: The cell of claim 9, wherein the cell has been isolated from bone marrow.

11: A composition comprising the lentiviral vector of claim 1 and a pharmaceutically acceptable carrier.

12: A composition comprising viral particles, wherein the viral particles comprise the lentiviral vector of claim 1.

13: A method of inhibiting, treating, and/or preventing multiple sulfatase deficiency (MSD), metachromatic leukodystrophy (MLD) and/or a sulfatase deficiency in a subject, said method comprising administering the lentiviral vector of claim 1 to the subject.

14: The method of claim 13, wherein said cells are selected from the group consisting of hematopoietic stem cells, bone marrow cells, microglia, and fibroblasts.

15: The method of claim 13, wherein the cells are isolated from the subject to be treated.

16: A method of inhibiting, treating, and/or preventing a disease or disorder in a subject, said method comprising administering the lentiviral vector of claim 1 to the subject.

17: The method of claim 16, wherein said disease or disorder is adrenoleukodystrophy (ALD) and the therapeutic protein is ATP binding cassette subfamily D member 1 (ABCD1).

18: A method of inhibiting, treating, and/or preventing multiple sulfatase deficiency (MSD), metachromatic leukodystrophy (MLD) and/or a sulfatase deficiency in a subject, said method comprising introducing the lentiviral vector of claim 1 into cells and delivering the cells to the subject.

19: A method of inhibiting, treating, and/or preventing a disease or disorder in a subject, said method comprising introducing the lentiviral vector of claim 1 into cells and delivering the cells to the subject.


SEQUENCE LISTING (17 sequences)

SEQ ID NO: 1 - protein, 509 aa, Homo sapiens
MSMGAPRSLLLALAAGLAVARPPNIVLIFADDLGYGDLGCYGHPSSTTPNLDQLAAGGLRFTDFYVPVSLCTPSRAALLTGRLPVRMGMYPGVLVPSSRGGLPLEEVTVAEVLAARGYLTGMAGKWHLGVGPEGAFLPPHQGFHRFLGIPYSHDQGPCQNLTCFPPATPCDGGCDQGLVPIPLLANLSVEAQPPWLPGLEARYMAFAHDLMADAQRQDRPFFLYYASHHTHYPQFSGQSFAERSGRGPFGDSLMELDAAVGTLMTAIGDLGLLEETLVIFTADNGPETMRMSRGGCSGLLRCGKGTTYEGGVREPALAFWPGHIAPGVTHELASSLDLLPTLAALAGAPLPNVTLDGFDLSPLLLGTGKSPRQSLFFYPSYPDEVRGVFAVRTGKYKAHFFTQGSAHSDTTADPACHASSSLTAHEPPLLYDLSKDPGENYNLLGGVAGATPEVLQALKQLQLLKAQLDAAVTFGPSQVARGEDPALQICCHPGCTPRPACCHCPDPHA

SEQ ID NO: 2 - DNA, 4294 bp, Homo sapiens
  source 1..4294: mol_type = other DNA
gaggcggaagcgcccgcagcccggtaccggctcctcctgggctccctctagcgccttccccccggcccgactccgctggtcagcgccaagtgacttacgcccccgaccctgagcccggaccgctaggcgaggaggatcagatctccgctcgagaatctgaaggtgccctggtcctggaggagttccgtcccagcccgcggtctcccggtactgtcgggccccggccctctggagcttcaggaggcggccgtcagggtcggggagtatttgggtccggggtctcagggaagggcggcgcctgggtctgcggtatcggaaagagcctgctggagccaagtagccctccctctcttgggacagacccctcggtcccatgtccatgggggcaccgcggtccctcctcctggccctggctgctggcctggccgttgcccgtccgcccaacatcgtgctgatctttgccgacgacctcggctatggggacctgggctgctatgggcaccccagctctaccactcccaacctggaccagctggcggcgggagggctgcggttcacagacttctacgtgcctgtgtctctgtgcacaccctctagggccgccctcctgaccggccggctcccggttcggatgggcatgtaccctggcgtcctggtgcccagctcccgggggggcctgcccctggaggaggtgaccgtggccgaagtcctggctgcccgaggctacctcacaggaatggccggcaagtggcaccttggggtggggcctgagggggccttcctgcccccccatcagggcttccatcgatttctaggcatcccgtactcccacgaccagggcccctgccagaacctgacctgcttcccgccggccactccttgcgacggtggctgtgaccagggcctggtccccatcccactgttggccaacctgtccgtggaggcgcagcccccctggctgcccggactagaggcccgctacatggctttcgcccatgacctcatggccgacgcccagcgccaggatcgccccttcttcctgtactatgcctctcaccacacccactaccctcagttcagtgggcagagctttgcagagcgttcaggccgcgggccatttggggactccctgatggagctggatgcagctgtggggaccctgatgacagccataggggacctggggctgcttgaagagacgctggtcatcttcactgcagacaatggacctgagaccatgcgtatgtcccgaggcggctgctccggtctcttgcggtgtggaaagggaacgacctacgagggcggtgtccgagagcctgccttggccttctggccaggtcatatcgctcccggcgtgacccacgagctggccagctccctggacctgctgcctaccctggcagccctggctggggccccactgcccaatgtcaccttggatggctttgacctcagccccctgctgctgggcacaggcaagagccctcggcagtctctcttcttctacccgtcctacccagacgaggtccgtggggtttttgctgtgcggactggaaagtacaaggctcacttcttcacccagggctctgcccacagtgataccactgcagaccctgcctgccacgcctccagctctctgactgctcatgagcccccgctgctctatgacctgtccaaggaccctggtgagaactacaacctgctggggggtgtggccggggccaccccagaggtgctgcaagccctgaaacagcttcagctgctcaaggcccagttagacgcagctgtgaccttcggccccagccaggtggcccggggcgaggaccccgccctgcagatctgctgtcatcctggctgcaccccccgcccagcttgctgccattgcccagatccccatgcctgagggcccctcggctggcctgggcatgtgatggctcctcactgggagcctgtgggggaggctcaggtgtctggagggggtttgtgcctgataacgtaataacaccagtggagacttgcagatgtgacaattcgtccaatcctggggtaatgctgtgtgctggtgccggtcccctgtggtacgaatgaggaaactgaggtgcagagaggttcaggacttgtacaagatcacccagccagaaagaggttgggctgggatttgaaccctggtgtcgtggctctggaagctgccctggcgccttggtgatctgcgtgggtcagtgcacacaggcacacgtcagcctcaaggacatgggcacatctgttcacaggagcagcgccacgtgcctttgagtgccaggaacggggtgggagggtgggagggtgtgagggccagaagactcagaagatgcaaagtgcctgagagagacgggatattcccccagaagaagcattcttagagacacaggcactggacctccttggttcttataagaaacctgtctgaagctgggtgatgagttgcacactccaggtggggctaaggggcctggagcccctgctggctcctaggaaggcacagcagcaggccctgagacggctcctctggggcccctccaccctcccaggcctctgcatttcacctgtgcccacacttctgtctcctgccttcaccttttgacccactactaacgattctccacccagcagacaaagtgatctcttaaaaatatctgttggctgggcacggtggctcacgcctgtaatcccagcactttaggaagccgaggcgggtggatcacctgaggtcgggagttcgagaccagcctgaccaacatggagaaaccccatctctactaaaaatacaaaattagccaggtgtagtggtgcatccctgtaatcccagctacttgggagtctgaggctggagaatcacttgaacctgggaggcggtggttgcagtgagccgagatcgcaccattgcactccagcctgggcaacaagagaaaaactctgtctcaaaaaacaaaaaatctgttaggctgcacacggcgattcactcctgtattcccagtgctttgggaggctgaggtgagaggatgcctgaggccaggaattcagaccagcctgggcaacatagtgagaccccagctctaaagatttgtttttgttttttttttttttttttttttttttttttttttttttttgagacggagtctcgctctgtcgcccaggctagagtgcagtggtaccatctccgctcactgcaacctccgcctcccgggttccagggattctcctgcctcagcctccctagtagctggaactacaggtgtgtgctgccatgcccagctaatttttttttatttaatagagacaagatttcaccatgttggccaggctggtctcaaactcctgacctcaggtgatccacccgcctcagcctcccaaagtgctgggattacaggtgtgaaccaccacacctggccaacaatatttgttttaattagccaggcgtggtagcatttgtcctagcaatttgggaggttgaggtgggagaatcacttcagcccactaggtcgaggctgtagtgagctataattgtaccactgcactccagcctcggggacagagtgagaccctgtctgcaaataaacaaataaaacatcaggctgggcttgagcatctattcctgctcaaaatttcgcaggcttctcagaagaaaatccaaaccccttacagtgacccagtttgcccttgaggcctccacccacacccccttccccccagtcttagggggtggcctggctgttcccttcaacggcaacgctctgcctccattgttggcctcctctgcagggagggactgtctgagcacctgcccgtgtctgtgcagcatggcacactgacgtcaggcccacgtgcatgcccaggtggccagtcacacgccaggtgctccctcagtgttggccaagtgagaggagcacaccttccgggcgttcagacacctccccgtggcagacaccgttcgttgctaccaaacagccacctccttcctaatgggctcccatttttcagtgctgggcaaaggtcccttgatcttggagttgcagcctctttctctccaaggagggcggtgaccagcctgagccagtcaatccagtgattggttcaggagtagcctgtgaccaggagtcctggtagtgaacgactggggcagccctgggggtgaggaccttgcgcagccgtcacaggccctgattggacactgggcagctgctaacccagtgtctccagctgcctacctggagagctccaagcgtaagaaaataaaccctgcctgttgaagcca

SEQ ID NO: 3 - DNA, 2238 bp, Homo sapiens
  source 1..2238: mol_type = other DNA
atgccggtgctctccaggccccggccctggcgggggaacacgctgaagcgcacggccgtgctcctggccctcgcggcctatggagcccacaaagtctaccccttggtgcgccagtgcctggccccggccaggggtcttcaggcgcccgccggggagcccacgcaggaggcctccggggtcgcggcggccaaagctggcatgaaccgggtattcctgcagcggctcctgtggctcctgcggctgctgttcccccgggtcctgtgccgggagacggggctgctggccctgcactcggccgccttggtgagccgcaccttcctgtcggtgtatgtggcccgcctggacggaaggctggcccgctgcatcgtccgcaaggacccgcgggcttttggctggcagctgctgcagtggctcctcatcgccctccctgctaccttcgtcaacagtgccatccgttacctggagggccaactggccctgtcgttccgcagccgtctggtggcccacgcctaccgcctctacttctcccagcagacctactaccgggtcagcaacatggacgggcggcttcgcaaccctgaccagtctctgacggaggacgtggtggcctttgcggcctctgtggcccacctctactccaacctgaccaagccactcctggacgtggctgtgacttcctacaccctgcttcgggcggcccgctcccgtggagccggcacagcctggccctcggccatcgccggcctcgtggtgttcctcacggccaacgtgctgcgggccttctcgcccaagttcggggagctggtggcagaggaggcgcggcggaagggggagctgcgctacatgcactcgcgtgtggtggccaactcggaggagatcgccttctatgggggccatgaggtggagctggccctgctacagcgctcctaccaggacctggcctcgcagatcaacctcatccttctggaacgcctgtggtatgttatgctggagcagttcctcatgaagtatgtgtggagcgcctcgggcctgctcatggtggctgtccccatcatcactgccactggctactcagagtcagatgcagaggccgtgaagaaggcagccttggaaaagaaggaggaggagctggtgagcgagcgcacagaagccttcactattgcccgcaacctcctgacagcggctgcagatgccattgagcggatcatgtcgtcgtacaaggaggtgacggagctggctggctacacagcccgggtgcacgagatgttccaggtatttgaagatgttcagcgctgtcacttcaagaggcccagggagctagaggacgctcaggcggggtctgggaccataggccggtctggtgtccgtgtggagggccccctgaagatccgaggccaggtggtggatgtggaacaggggatcatctgcgagaacatccccatcgtcacgccctcaggagaggtggtggtggccagcctcaacatcagggtggaggaaggcatgcatctgctcatcacaggccccaatggctgcggcaagagctccctgttccggatcctgggtgggctctggcccacgtacggtggtgtgctctacaagcccccaccccagcgcatgttctacatcccgcagaggccctacatgtctgtgggctccctgcgtgaccaggtgatctacccggactcagtggaggacatgcaaaggaagggctactcggagcaggacctggaagccatcctggacgtcgtgcacctgcaccacatcctgcagcgggagggaggttgggaggctatgtgtgactggaaggacgtcctgtcgggtggcgagaagcagagaatcggcatggcccgcatgttctaccacaggcccaagtacgccctcctggatgaatgcaccagcgccgtgagcatcgacgtggaaggcaagatcttccaggcggccaaggacgcgggcattgccctgctctccatcacccaccggccctccctgtggaaataccacacacacttgctacagttcgatggggagggcggctggaagttcgagaagctggactcagctgcccgcctgagcctgacggaggagaagcagcggctggagcagcagctggcgggcattcccaagatgcagcggcgcctccaggagctctgccagatcctgggcgaggccgtggccccagcgcatgtgccggcacctagcccgcaaggccctggtggcctccagggtgcctccacctga

SEQ ID NO: 4 - DNA, 2238 bp, synthetic construct
  source 1..2238: mol_type = other DNA
atgccggtgctctccaggccccggccctggcgggggaacacgctgaagcgcacggccgtgctcctggccctcgcggcctatggagcccacaaagtctaccccttggtgcgccagtgcctggccccggccaggggtcttcaggcgcccgccggggagcccacgcaggaggcctccggggtcgcggcggccaaagctggcatgaaccgggtattcctgcagcggctcctgtggctcctgcggctgctgttcccccgggtcctgtgccgggagacggggctgctggccctgcactcggccgccttggtgagccgcacttttctgtcagtgtatgtggcccgcctggacggaaggctggcccgctgcatcgtccgcaaggacccgcgggcttttggctggcagctgctgcagtggctcctcatcgccctccctgctaccttcgtcaacagtgccatccgttacctggagggccaactggccctgtcgttccgcagccgtctggtggcccacgcctaccgcctctacttctcccagcaaacctattatcgggtcagcaacatggacgggcggcttcgcaaccctgaccagtctctgacggaggacgtggtggcctttgcggcctctgtggcccacctctactccaacctgaccaagccactcctggacgtggctgtgacttcctacaccctgcttcgggcggcccgctcccgtggagccggcacagcctggccctcggccatcgccggcctcgtggtgttcctcacggccaacgtgctgcgggccttctcgcccaagttcggggagctggtggcagaggaggcgcggcggaagggggagctgcgctacatgcactcgcgtgtggtggccaactcggaggagatcgccttctatgggggccatgaggtggagctggccctgctacagcgctcctaccaggacctggcctcgcagatcaacctcatccttctggaacgcctgtggtatgttatgctggagcagttcctcatgaagtatgtgtggagcgcctcgggcctgctcatggtggctgtccccatcatcactgccactggctactcagagtcagatgcagaggccgtgaagaaggcagccttggaaaagaaggaggaggagctggtgagcgagcgcacagaagccttcactattgcccgcaacctcctgacagcggctgcagatgccattgagcggatcatgtcgtcgtacaaggaggtgacggagctggctggctacacagcccgggtgcacgagatgttccaggtatttgaagatgttcagcgctgtcacttcaagaggcccagggagctagaggacgctcaggcggggtctgggaccataggccggtctggtgtccgtgtggagggccccctgaagatccgaggccaggtggtggatgtggaacaggggatcatctgcgagaacatccccatcgtcacgccctcaggagaggtggtggtggccagcctcaacatcagggtggaggaaggcatgcatctgctcatcacaggccccaatggctgcggcaagagctccctgttccggatcctgggtgggctctggcccacgtacggtggtgtgctctacaagcccccaccccagcgcatgttctacatcccgcagaggccctacatgtctgtgggctccctgcgtgaccaggtgatctacccggactcagtggaggacatgcaaaggaagggctactcggagcaggacctggaagccatcctggacgtcgtgcacctgcaccacatcctgcagcgggagggaggttgggaggctatgtgtgactggaaggacgtcctgtcgggtggcgagaagcagagaatcggcatggcccgcatgttctaccacaggcccaagtacgccctcctggatgaatgcaccagcgccgtgagcatcgacgtggaaggcaagatcttccaggcggccaaggacgcgggcattgccctgctctccatcacccaccggccctccctgtggaaataccacacacacttgctacagttcgatggggagggcggctggaagttcgagaagctggactcagctgcccgcctgagcctgacggaggagaagcagcggctggagcagcagctggcgggcattcccaagatgcagcggcgcctccaggagctctgccagatcctgggcgaggccgtggccccagcgcatgtgccggcacctagcccgcaaggccctggtggcctccagggtgcctccacctga

SEQ ID NO: 5 - DNA, 616 bp, Woodchuck hepatitis virus
  source 1..616: mol_type = other DNA
gtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacc

SEQ ID NO: 6 - DNA, 190 bp, synthetic construct
  source 1..190: mol_type = other DNA
gtgcgggccaggcccccgagggccttatcggccccagaggcgcttgctgtcgggccgggcgctcccggcacgggcgggcggaggggtggcgcccgcctggggaccgcagattacaagagcacctcctcccccaaccccaggaggccccgctccccaggcctcggccggcgcggacccctggttgccccgg

SEQ ID NO: 7 - DNA, 36 bp, synthetic construct
  source 1..36: mol_type = other DNA
aagggagacatctagtgatataagtgtgaactacac

SEQ ID NO: 8 - protein, 374 aa, Homo sapiens
MAAPALGLVCGRCPELGLVLLLLLLSLLCGAAGSQEAGTGAGAGSLAGSCGCGTPQRPGAHGSSAAAHRYSREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMD

SEQ ID NO: 9 - DNA, 2147 bp, Homo sapiens
  source 1..2147: mol_type = other DNA
gggtcacatggcccgcgggacaacatggctgcgcccgcactagggctggtgtgtggacgttgccctgagctgggtctcgtcctcttgctgctgctgctctcgctgctgtgtggagcggcagggagccaggaggccgggaccggtgcgggcgcggggtcccttgcgggttcttgcggctgcggcacgccccagcggcctggcgcccatggcagttcggcagccgctcaccgatactcgcgggaggctaacgctccgggccccgtacccggagagcggcaactcgcgcactcaaagatggtccccatccctgctggagtatttacaatgggcacagatgatcctcagataaagcaggatggggaagcacctgcgaggagagttactattgatgccttttacatggatgcctatgaagtcagtaatactgaatttgagaagtttgtgaactcaactggctatttgacagaggctgagaagtttggcgactcctttgtctttgaaggcatgttgagtgagcaagtgaagaccaatattcaacaggcagttgcagctgctccctggtggttacctgtgaaaggcgctaactggagacacccagaagggcctgactctactattctgcacaggccggatcatccagttctccatgtgtcctggaatgatgcggttgcctactgcacttgggcagggaagcggctgcccacggaagctgagtgggaatacagctgtcgaggaggcctgcataatagacttttcccctggggcaacaaactgcagcccaaaggccagcattatgccaacatttggcagggcgagtttccggtgaccaacactggtgaggatggcttccaaggaactgcgcctgttgatgccttccctcccaatggttatggcttatacaacatagtggggaacgcatgggaatggacttcagactggtggactgttcatcattctgttgaagaaacgcttaacccaaaaggtcccccttctgggaaagaccgagtgaagaaaggtggatcctacatgtgccataggtcttattgttacaggtatcgctgtgctgctcggagccagaacacacctgatagctctgcttcgaatctgggattccgctgtgcagccgaccgcctgcccactatggactgacaaccaaggaaagtcttccccagtccaaggagcagtcgtgtctgacctacattgggcttttctcagaactttgaacgatcccatgcaaagaattcccaccctgaggtgggttacatacctgcccaatggccaaaggaaccgccttgtgagaccaaattgctgacctgggtcagtgcatgtgctttatggtgtggtgcatctttggagatcatcaccatattttacttttgagagtctttaaagaggaaggggagtggagggaaccctgagctaggcttcaggaggcccgcgtcctacgcaggctctgccacaggggttagaccccaggtccgacgcttgaccttcctgggcctcaagtgccctcccctatcaaatgaagggatggacagcatgacctctgggtgtctctccaactcaccagttctaaaaagggtatcagattctattgtgacttcatagtgagaatttattatagattattttttagctattttttccatgtgtgaaccttgagtgatactaatcatgtaaagtaagagttctcttatgtattattttcggaagaggggtgtggtgactcctttatattcgtactgcactttgtttttccaaggaaatcagtgtcttttacgttgttatgatgaatcccacatggggccggtgatggtatgctgaagttcagccgttgaacacataggaatgtctgtggggtgactctactgtgctttatcttttaacattaagtgcctttggttcagaggggcagtcataagctctgtttccccctctccccaaagccttcagcgaacgtgaaatgtgcgctaaacggggaaacctgtttaattctagatatagggaaaaaggaacgaggaccttgaatgagctatattcagggtatccggtattttgtaatagggaataggaaaccttgttggctgtggaatatccgatgctttgaatcatgcactgtgttgaataaacgtatctgctaaatcagg

SEQ ID NO: 10 - DNA, 1184 bp, Homo sapiens
  source 1..1184: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgag

SEQ ID NO: 11 - DNA, 420 bp, Homo sapiens
  source 1..420: mol_type = other DNA
gtacaggggtcagcccagaggtccaggggaaaggagtggaaaccgatttccccaccaagggaggggcctgtacctcagctgttcccatagctacttgccacaactgccaagcaagtttcgctgagtttgacacatggatccctgtggatcaactgccctaggactccgtttgcacccatgtgacactgttgactttgccctgatgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagcc

SEQ ID NO: 12 - DNA, 570 bp, synthetic construct
  source 1..570: mol_type = other DNA
gtacaggggtcagcccagaggtccaggggaaaggagtggaaaccgatttccccaccaagggaggggcctgtacctcagctgttcccatagctacttgccacaactgccaagcaagtttcgctgagtttgacacatggatccctgtggatcaactgccctaggactccgtttgcacccatgtgacactgttgactttgccctgatgaagcagggccaacagtcccctaacttaattacaaaaactaatgactaagagagaggtggctagagctgaggcccctgagtcaggctgtgggtgggatcatctccagtacaggaagtgagactttcatttcctcctttccaagagagggctgagggagcagggttgagcaactggtgcagacagcctagctggactttgggtgaggcggttcagcctaaaggctggctgtgcttttctcgggggccctgctggggctactggcaggtaaggaggaaggaggctgaggggagggggcccctgggagggagcctgccctgggttgctaaccatctcctctctgccaaaagcccaggggacagggaatg

SEQ ID NO: 13 - DNA, 1530 bp, synthetic construct
  source 1..1530: mol_type = other DNA
atgagtatgggcgcaccacgatcactgctgctggcactggctgctggactggctgtggcacgaccacctaatattgtgctgattttcgcagacgatctgggatacggcgacctgggatgctatggccacccaagctccaccacacccaacctggaccagctggcagcaggaggcctgcggttcaccgacttctacgtgccagtgagcctgtgcaccccatccagagccgccctgctgacaggcaggctgccagtgcgcatgggcatgtatcctggcgtgctggtgccatctagcaggggaggcctgccactggaggaggtgaccgtggcagaggtgctggcagcaaggggctacctgacaggaatggccggcaagtggcacctgggagtgggaccagagggagccttcctgccccctcaccagggcttccaccggtttctgggcatcccttattctcacgaccagggcccatgccagaacctgacctgttttccaccagcaacaccatgcgacggaggatgtgatcagggcctggtgccaatcccactgctggcaaatctgagcgtggaggcacagcctccatggctgcctggcctggaggcaagatacatggccttcgcccacgacctgatggcagatgcacagcggcaggatagacctttctttctgtactatgcctcccaccacacccactatccacagttcagcggccagtcctttgccgagaggtccggaaggggaccattcggcgactctctgatggagctggatgccgccgtgggcaccctgatgacagcaatcggcgacctgggcctgctggaggagacactggtcatcttcaccgccgataacggccctgagacaatgcggatgtctagaggaggatgcagcggcctgctgagatgtggcaagggaaccacatacgagggaggcgtgcgcgagcctgccctggcattttggccaggacacatcgcacctggagtgacccacgagctggcatcctctctggacctgctgccaacactggccgccctggcaggagcacctctgccaaatgtgaccctggacggcttcgatctgagcccactgctgctgggaaccggcaagtcccctaggcagtctctgttcttttacccctcctatcctgatgaggtgcggggcgtgtttgccgtgagaaccggcaagtacaaggcccacttctttacacagggctctgcccacagcgacaccacagccgatcccgcatgccacgcaagctcctctctgaccgcacacgagccacctctgctgtacgacctgtccaaggaccccggcgagaactataatctgctgggaggagtggcaggagcaacccctgaggtgctgcaggccctgaagcagctgcagctgctgaaggcacagctggacgcagcagtgacattcggcccaagccaggtggcaagaggagaggaccccgccctgcagatctgttgtcatcctggctgtactcctagacctgcttgctgtcattgtcctgatcctcacgcataa

SEQ ID NO: 14 - DNA, 618 bp, synthetic construct
  source 1..618: mol_type = other DNA
tcggtaggaattaattccgccccccccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgattgctatatggccacaacc

SEQ ID NO: 15 - DNA, 12 bp, synthetic construct
  source 1..12: mol_type = other DNA
taggccgccacc

SEQ ID NO: 16 - DNA, 1125 bp, Homo sapiens
  source 1..1125: mol_type = other DNA
atggctgcgcccgcactagggctggtgtgtggacgttgccctgagctgggtctcgtcctcttgctgctgctgctctcgctgctgtgtggagcggcagggagccaggaggccgggaccggtgcgggcgcggggtcccttgcgggttcttgcggctgcggcacgccccagcggcctggcgcccatggcagttcggcagccgctcaccgatactcgcgggaggctaacgctccgggccccgtacccggagagcggcaactcgcgcactcaaagatggtccccatccctgctggagtatttacaatgggcacagatgatcctcagataaagcaggatggggaagcccccgcaagaagggtgactattgatgccttttacatggatgcctatgaagtcagtaatactgaatttgagaagtttgtgaactcaactggctatttgacagaggctgagaagtttggcgactcctttgtctttgagggaatgctcagcgaacaagtgaagaccaatattcaacaggcagttgcagctgctccctggtggttacctgtgaaaggcgctaactggagacacccagaagggcctgactctactattctgcacaggccggatcatccagttctccatgtgtcctggaatgatgcggttgcctactgcacttgggcagggaagcggctgcccacggaagctgagtgggaatacagctgtcgaggaggcctgcataatagacttttcccctggggcaacaaactgcagcccaaaggccagcattatgccaacatttggcagggcgagtttccggtgaccaacactggtgaggatggcttccaaggaactgcgcctgttgatgccttccctcccaatggttatggcttatacaacatagtggggaacgcatgggaatggacttcagactggtggactgttcatcattctgttgaagaaacgcttaacccaaaaggtcccccttctgggaaagaccgagtgaagaaaggtggatcctacatgtgccataggtcttattgttacaggtatcgctgtgctgctcggagccagaacacacctgatagctctgcttcgaatctgggattccgctgtgcagccgaccgcctgcccactatggactga

SEQ ID NO: 17 - DNA, 9854 bp, synthetic construct
  source 1..9854: mol_type = other DNA
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatgtatacgtgcgggccaggcccccgagggccttatcggccccagaggcgcttgctgtcgggccgggcgctcccggcacgggcgggcggaggggtggcgcccgcctggggaccgcagattacaagagcacctcctcccccaaccccaggaggccccgctccccaggcctcggccggcgcggacccctggttgccccgggatatcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggggatccatgagtatgggcgcaccacgatcactgctgctggcactggctgctggactggctgtggcacgaccacctaatattgtgctgattttcgcagacgatctgggatacggcgacctgggatgctatggccacccaagctccaccacacccaacctggaccagctggcagcaggaggcctgcggttcaccgacttctacgtgccagtgagcctgtgcaccccatccagagccgccctgctgacaggcaggctgccagtgcgcatgggcatgtatcctggcgtgctggtgccatctagcaggggaggcctgccactggaggaggtgaccgtggcagaggtgctggcagcaaggggctacctgacaggaatggccggcaagtggcacctgggagtgggaccagagggagccttcctgccccctcaccagggcttccaccggtttctgggcatcccttattctcacgaccagggcccatgccagaacctgacctgttttccaccagcaacaccatgcgacggaggatgtgatcagggcctggtgccaatcccactgctggcaaatctgagcgtggaggcacagcctccatggctgcctggcctggaggcaagatacatggccttcgcccacgacctgatggcagatgcacagcggcaggatagacctttctttctgtactatgcctcccaccacacccactatccacagttcagcggccagtcctttgccgagaggtccggaaggggaccattcggcgactctctgatggagctggatgccgccgtgggcaccctgatgacagcaatcggcgacctgggcctgctggaggagacactggtcatcttcaccgccgataacggccctgagacaatgcggatgtctagaggaggatgcagcggcctgctgagatgtggcaagggaaccacatacgagggaggcgtgcgcgagcctgccctggcattttggccaggacacatcgcacctggagtgacccacgagctggcatcctctctggacctgctgccaacactggccgccctggcaggagcacctctgccaaatgtgaccctggacggcttcgatctgagcccactgctgctgggaaccggcaagtcccctaggcagtctctgttcttttacccctcctatcctgatgaggtgcggggcgtgtttgccgtgagaaccggcaagtacaaggcccacttctttacacagggctctgcccacagcgacaccacagccgatcccgcatgccacgcaagctcctctctgaccgcacacgagccacctctgctgtacgacctgtccaaggaccccggcgagaactataatctgctgggaggagtggcaggagcaacccctgaggtgctgcaggccctgaagcagctgcagctgctgaaggcacagctggacgcagcagtgacattcggcccaagccaggtggcaagaggagaggaccccgccctgcagatctgttgtcatcctggctgtactcctagacctgcttgctgtcattgtcctgatcctcacgcataagtcgactttaagaccaatgacttacaaggcagctgtagatcttagccactttttaaaagaaaaggggggactggaagggctaattcactcccaacgaagacaagatctgctttttgctgtgtagttcacacttatatcactagatgtctcccttactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcaggctagcaaacaaaagatctagaaagaaacaaacccgggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggaaagaaccagctggggctcgagatccactagttctagcctcgaggctagagcggccgccaccgcggtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc